(19) United States
(12) Patent Application Publication
(10) Pub. No.: US 2025/0163380 A1
(43) Pub. Date: May 22, 2025

(54) A COMBINED HUMAN GASTRULOID MODEL OF CARDIOGENESIS AND NEUROGENESIS

(71) Applicant: The Research Foundation for the State University of New York, Albany, NY (US)

(72) Inventors: Janet PALUH, Albany, NY (US); Zachary OLMSTED, Oneida, NY (US)

(21) Appl. No.: 18/839,613

(22) PCT Filed: Feb. 20, 2023
(86) PCT No.: PCT/US23/13415

§ 371 Date: Aug. 19, 2024

Related U.S. Application Data
(60) Provisional application No. 63/311,498, filed on Feb. 18, 2022.
(60) Provisional application No. 63/419,507, filed on Oct. 26, 2022.

(51) Int. Cl. C12N 5/077

(52) U.S. Cl. CPC C12N 5/0657; C12N 2500/38; C12N 2501/105; C12N 2501/115; C12N 2501/12; C12N 2501/165; C12N 2506/1307

(57) ABSTRACT

The preset disclosure relates to EMLOC gastruloids and methods of generating elongating multi-lineage organized cardiac (EMLOC) gastruloids from pluripotent stem cells, including: contacting a colony of human induced pluripotent stem cells with hepatocyte growth factor (HGF), insulin-like growth factor-1 (IGF-1), fibroblast growth factor-2 (FGF-2) under culture conditions for a first duration to form a first plurality of gastruloids; and contacting the first plurality of gastruloids with vascular endothelial growth factor (VEGF), fibroblast growth factor-2 (FGF-2), and ascorbic acid under culture conditions for a second duration to form EMLOC gastruloids.

### CROSS-REFERENCE TO RELATED APPLICATIONS

The present application claims the benefit of prior-filed U.S. Provisional Application Ser. No. 63/311,498 that was filed on Feb. 18, 2022 and U.S. Provisional Application Ser. No. 63/419,507 that was filed on Oct. 26, 2022, the disclosure of both these applications is hereby incorporated by reference.

### FIELD OF THE INVENTION

This disclosure generally relates to a method of generating one or more EMLOC gastruloids, one or more EMLOC gastruloid compositions, and/or a combined human gastruloid model of cardiogenesis and neurogenesis. In some embodiments, the disclosure provides compositions and methods employing stem cell-derived heart-like structures. In some embodiments, methods of generating heart-like tissues from human stem cells and the resulting tissues are provided. In some embodiments, uses of such tissues for research, compound screening and analysis, and therapeutics are provided.

### BACKGROUND

The complex nature of in vivo cardiogenesis underlies the difficulties in establishing in vitro cardiac developmental models with human cells. The heart is the first organ to form in the mammalian embryo, caudal to the embryonic brain and within the developing trunk. It becomes contractile as a tube prior to complex morphogenesis into septated chambers and co-developmental population by neurons for innervation (Harvey, 2002; Hasan, 2013). In order to accommodate both contractility and structural rearrangement, the developing heart undergoes alternating phases of cardiac differentiation and morphogenesis (Ivanovitch et al., 2017). Calcium handling properties become refined during cardiac differentiation (Tyser et al., 2016). The cardiac crescent is the first bilateral structure to form and precedes epithelialization and formation of the transversal heart tube. At this stage, the heart tube remains open at the dorsal aspect, bound by dorsal mesocardium, and then seals during formation of the closed linear heart tube and outflow tracts. Intrinsic cell-driven forces within the tube and extrinsic physical constraints are known to mediate the establishment of left-right asymmetries required for heart function (Desgrange et al., 2018). Such complexity in cardiogenesis lays the framework for lifelong functioning of the adult heart, but also underlies the propensity for congenital heart disease in humans where developmental errors induce cardiac malformations (van der Linde et al., 2011; Desgrange et al., 2018). The ability to generate in vitro models of heart development that mimic essential aspects of multi-lineage input to cardiogenesis is needed to benefit biomedical treatments of heart disease and progress towards ex vivo organogenesis.

Organoid technology is revolutionizing the study of human development and disease, recapitulating key aspects of spatiotemporal tissue morphogenesis (Clevers, 2016; Olmsted and Paluh, 2021c). Most current organoid technologies are directed towards single tissue endpoints that lack the cellular contextual diversity present in normal organogenesis through inductive and mechanical interactions. As such, the ability to generate organotypic human cardiac organoids that form according to the in situ developmental signaling blueprint and integrate with the developing nervous system has not been achieved. The existing human cardiac organoid models derive primarily from pre-differentiated cardiomyocytes and their spheroid aggregates that form irrespective of developmental timelines (Nguyen et al., 2014; Giacomelli et al., 2017; Polonchuk et al., 2017; Andersen et al., 2018), or models that rely on integrated bioengineering efforts to constrain morphogenetic patterning (Ma et al., 2015; Lind et al., 2017; Macqueen et al., 2018; Hookway et al., 2019). These models lack identified critical inductive tissues indispensable to natural heart development such as the foregut, described as a central organizer of cardiogenesis in multiple species and acting through both inductive and structural interactions between endoderm and splanchnic mesoderm (Nascone and Mercola, 1995; Schultheiss et al., 1995; Varner and Taber, 2012; Anderson et al., 2016; Kidokoro et al., 2018; Han et al., 2020).

Two recent studies with human iPSCs succeeded in the co-production of cardiac and gastrointestinal tissue in single organoids without organized chambers (Silva et al., 2021; Drakhlis et al., 2021). As well, Hofbauer et al. (2021) succeeded in generating self-organized, isolated cardioids exhibiting chamber-like structures from human pluripotent stem cells that were used to model cardiac injury (Hofbauer et al., 2021). Although important advances to the cardiogenesis field, neural cells were not co-generated in these systems and were absent. One murine study generated chambered cardiac organoids from mESCs by embedding in exogenous extracellular matrix (ECM) with supplied FGF4 (Lee et al., 2020). More recently, Rossi et al. (2021) used mESC-derived gastruloids to recapitulate aspects of early cardiogenesis including first and second heart field contributions without extracellular matrix (ECM) embedding. Gastruloid research has been broadly applicable for conducting multi-lineage interaction studies in the trunk (van den Brink et al., 2014; van den Brink et al., 2020; Veenvliet et al., 2020; Olmsted and Paluh, 2021a). However, no study with human cells has succeeded in generating a de novo model to recapitulate cardiogenesis in an embryo-like, multi-lineage context and, in particular, with neuronal cooperative development that is a vital functional component. What is needed are human gastruloids that capture numerous key developmental aspects of human cardiogenesis and neurogenesis along with endoderm-derived primitive gut tube and other lineages.

A unique human trunk model system referred to as elongating multi-lineage organized (EMLO) gastruloids (Olmsted and Paluh, 2021a, 2021b) has been described (See e.g., Olmsted, Z. T., Paluh, J. L. Co-development of central and peripheral neurons with trunk mesendoderm in human elongating multi-lineage organized gastruloids. Nat Commun 12, 3020 (2021) https://doi.org/10.1038/s41467-021-23294-7) (herein incorporated entirely by reference). Neural crest lineage in EMLOs reveals insights into enteric development and the formation of the enteric nervous system. The enteric multi-lineage niche in EMLOs achieved only limited embryonic cardiogenesis that included generation of cardiomyocytes anterior to the gut tube. What is needed is extended or improved embryonic cardiogenesis.

### SUMMARY

In embodiments, the present disclosure includes a method of generating elongating multi-lineage organized cardiac (EMLOC) gastruloids, including: contacting a colony of human induced pluripotent stem cells with hepatocyte growth factor (HGF), insulin-like growth factor-1 (IGF-1), fibroblast growth factor-2 (FGF-2) under culture conditions for a first duration to form a first plurality of gastruloids; and contacting the first plurality of gastruloids with vascular endothelial growth factor (VEGF), fibroblast growth factor-2 (FGF-2), and ascorbic acid under culture conditions for a second duration to form EMLOC gastruloids. In embodiments, the growth factor (HGF), insulin-like growth factor (IGF), fibroblast growth factor (FGF) are provided in an effective amount to coax the colony of human induced pluripotent stem cells to form a first plurality of gastruloids. In embodiments, the vascular endothelial growth factor (VEGF), fibroblast growth factor (FGF), and ascorbic acid are provided in an effective amount to coax the first plurality of gastruloids to form EMLOC gastruloids. In embodiments, the human induced pluripotent stem cells are contacted under culture conditions with 2 ng/ml hepatocyte growth factor (HGF), 2 ng/ml insulin-like growth factor (IGF), 10 ng/ml fibroblast growth factor (FGF) for a first duration, and 5 ng/ml vascular endothelial growth factor (VEGF), 30 ng/ml fibroblast growth factor (FGF), and 0.5 mM ascorbic acid for a second duration. In embodiments, the contacting occurs in an environment characterized as cardiac permissive. In embodiments, the colony of human induced pluripotent stem cells is characterized as trunk-biased stem cells. In embodiments, the first duration is 24 hours, and the second duration is 2-7 days. In embodiments, the colony of human induced pluripotent stem cells are trunk-biased stem cells.

In some embodiments, the present disclosure relates to one or more EMLOC gastruloids formed by one or more methods of the present disclosure. In embodiments, the present disclosure includes one or more EMLOC gastruloids, including a three-dimensional structure including or consisting of contractile innervated tissue. In some embodiments, an EMLOC gastruloid of the present disclosure is characterized by having human heart characteristics. In some embodiments, an EMLOC gastruloid of the present disclosure mimics human heart at natural human development timepoints. In embodiments, an EMLOC gastruloid of the present disclosure expresses human heart genes at development timepoints substantially similar to natural human heart at substantially similar timepoints.

In some embodiments, the present disclosure relates to one or more EMLOC gastruloids formed by one or more methods of the present disclosure. In embodiments, the present disclosure includes one or more EMLOC gastruloids, including a three-dimensional structure including or consisting of contractile innervated tissue. In some embodiments, an EMLOC gastruloid of the present disclosure is characterized by having human heart characteristics. In some embodiments, an EMLOC gastruloid of the present disclosure mimics human heart at natural human development timepoints. In embodiments, an EMLOC gastruloid of the present disclosure expresses human heart genes at natural human development timepoints.

In embodiments, the present disclosure includes a method of generating a contractile innervated human heart tissue or organ, including: coaxing or inducing an exogenous population of stem cells to divide and differentiate to an innervated cardiac fate by contacting the exogenous population of stem cells with hepatocyte growth factor (HGF), insulin-like growth factor-1 (IGF-1), fibroblast growth factor-2 (FGF-2) under culture conditions for a first duration to form a first plurality of gastruloids; and contacting the first plurality of gastruloids with vascular endothelial growth factor (VEGF), fibroblast growth factor-2 (FGF-2), and ascorbic acid under culture conditions for a second duration.

In embodiments, the present disclosure includes a method of generating an organized co-developed neuro-cardiac gastruloid, including: contacting an exogenous population of stem cells with an effective amount of hepatocyte growth factor (HGF), insulin-like growth factor (IGF-1), fibroblast growth factor (FGF-2) for a first duration, and subsequently contacting the exogenous population of stem cells with an effective amount of vascular endothelial growth factor (VEGF), fibroblast growth factor (FGF-2), and ascorbic acid for a second duration. In embodiments, the present disclosure includes methods for utilizing dissociated cells of the elongating multi-lineage organized cardiac (EMLOC) gastruloids and reseeding the dissociated cells onto mammalian scaffold material to initiate structures similar in EMLOCs for cardiogenesis or innervation. In embodiments, the present disclosure includes methods for utilizing the elongating multi-lineage organized cardiac (EMLOC) gastruloids with mammalian cells and initiating structures similar in EMLOCs for cardiogenesis or innervation. In embodiments, the exogenous population of stem cells are characterized as adherent and/or induced by contacting the cells with a biologically active stem cell differentiation and reprogramming reagent and a fibroblast growth factor 2 (FGF-2).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1. EMLOC gastruloids capture cell type diversity of human cardiogenesis and trunk development. FIG. 1A overview of protocol for EMLOC gastruloid generation. Cardiogenesis was induced at 48 h post-aggregation by addition of VEGF and ascorbic acid (AA). FIG. 1B Immunofluorescence of day 7 H3.1.1 EMLOC immunostained for TUJ1 (red), cTnT (magenta), FOXA2 (cyan) and counterstained with DAPI (grey). Inset is high magnification Z-slice of FOXA2 foregut (fg) initialization. FIG. 1B indicates anterior (A) to posterior (P) orientation. FIG. 3C-3D reconstruction of anterior cardiac region from FIG. 1B with TUJ1 (red), cTnT (magenta) and DAPI (grey). The EMLOC chamber surface (left) and core (right) are shown. Individual scale bars provided. FIG. 1D UMAP visualization of ten annotated clusters from the integrated scRNAseq dataset containing day 7 (1,004 cells) and day 16 (1,855 cells) time points in EMLOC formation (2,859 total cells). FIG. 1E PHATE visualization from the integrated dataset shown in FIG. 1D. Cell lineages are labeled along with upregulated DEGs. FIG. 1 F Heatmap of the top five DEGs for each cluster of the integrated scRNAseq dataset. Abbreviations: anterior foregut (AFG), cardiac fibroblast (CF), cardiomyocyte (CM), epicardial cells (EP), first heart field (FHF), genitourinary (GU), intermediate mesoderm (IM), mitotic (mit), neuronal (N), outflow tract (OFT), second heart field (SHF), splanchnic mesoderm (SM), vascular endothelial cells (VE).

FIG. 2. Diverging lineages in EMLOCs advance according to in vivo developmental principles. FIG. 2A day 16 EMLOC scRNAseq dataset visualized by PHATE. Superimposed developmental lineages include cardiac (TNNT2 or LHX9), anterior foregut (FOXA2 and NKX2-1), epithelial (PAX2 and PAX8), and neural (FABP7 or STMN2). FIG. 2B signaling pathways segregate along diverging lineages in EMLOCs. BMP4 vs. BMP7 vs. SHH is shown (left) along with WNT2B vs. WNT1. FIG. 2C preserved cadherin and Hox codes in EMLOCs. CDH1 vs. CDH6 vs. CDH11 is shown (left) along with HOXA4 vs. HOXC9 vs. HOXD8 (right). FIGS. 2D-2E day 16 EMLOC scRNAseq dataset visualized by UMAP. FIG. 2D genes for sarcomere proteins involved in contractility (TNNT2, TNNT1, MYL7) are upregulated primarily in cluster 3. Biomarkers for FHF (TBX5) and SHF (HAND2) are shown. FIG. 2E epicardial cell and cardiac fibroblast biomarkers LHX2, LHX9, WT1 are upregulated in clusters 2 and 7. Cell proliferation marker MK167 depicts fewer mitotic cells in cluster 7 vs. cluster 2.

FIG. 3. Spontaneous contractility and calcium handling in the EMLOC cardiac region. FIG. 3A sarcomeres are visible with cTnT at day 7 in EMLOC formation. Progressive zoom with high magnification black and white image (right). FIG. 3B percentage of EMLOCs with beating chambers versus trunk biased gastruloids formed by the original EMLO protocol (Olmsted and Paluh, 2021a, 2021b). (N=3 repeat experiments; ***p=0.0005, t=10.40 df=4 by unpaired two-tailed t-test). FIG. 3C fluo-4 AM calcium imaging time course in two adjacent H3.1.1 EMLOCs. Individual scale bars provided. Images shown with fire LUT (ImageJ) and calibration bar. Individual scale bars provided. FIG. 3D quantified F/Fₒ time series from FIG. 3C. The two EMLOCs shown were captured in the same field and are representative of the population. FIG. 3E Box-and-whisker plot quantification of Fluo-4 AM Ca²⁺ spikes/min in H3.1.1 day 7 EMLOCs (n=10 EMLOCs, max=19, min=5, median=10.5, q1=8, q3=12.75) from N=2 separate differentiations. FIG. 3F genes involved in rapid ventricular conduction (IRX3), repolarization (IRX5), calcium flux (ITPR2) and handling (SLC8A1/NAC1) are upregulated in EMLOCs. Day 16 scRNAseq data visualized by PHATE.

FIG. 4. EMLOCs recapitulate early polarized heart tube formation events. FIG. 4A cartoon schematic of the embryo depicting anterior cardiac crescent (cc, red/white), foregut cavity (fg, black), and neural tube (nt, dark blue). Anterior-posterior axis is indicated. The bilateral cardiac crescent fuses to form the transversal heart tube (HT), dorsally open heart tube, and linear heart tube (closed). FIG. 4B Percentage of day 4 gastruloids with cardiac crescent using the original EMLO protocol (Olmsted and Paluh, 2021a, 2021b) versus the optimized EMLOC protocol (N=3 repeat experiments; ***p=0.0007, t=9.474, df=4 by unpaired two-tailed t-test). FIG. 4C day 4 EMLOCs exhibit cTnT+ cardiac crescent-like structures (magenta, top) with GATA6+ nuclei (bottom, cyan). Phase image of day 4 EMLOC is shown with labeled nt, fg, and cc corresponding structures (top right). FIG. 4D comparison of cTnT+ cardiogenic regions in day 4 versus day 5 EMLOCs. Two adjacent EMLOCs are shown for each time point. FIG. 4E high magnification of cardiac crescent structure with cTnT (magenta) and GATA6 (cyan). Individual channels are shown without pseudocolor. FIG. 4F immunofluorescence of cTnT (magenta) and laminin (cyan) with inverted cTnT channels depicts developing heart tube-like structure in EMLOCs (day 6). Z-slices and cTnT maximal projections are shown. FIG. 4G cardiac chamber precursors in two separate EMLOCs (day 7). Z-slices and cTnT maximal projections are shown. Individual scale bars provided.

FIG. 5. Cardiogenesis occurs anterior to gut tube endoderm. FIG. 5A left: cartoon schematic of foregut and heart tube developmental cross-section with dorsal mesocardium (DM). The dorsal-ventral (D-V) and right-left (R-L) embryonic axes are shown. Right: cardiac crescent differentiation microenvironment. Cardiomyocyte progenitors (red), endocardium (tan), and definitive endoderm (blue) are shown. FIG. 5B biomarker distribution using cTnT (cardiac, pink) and FOXA2 (gut tube, blue) in day 4 EMLOC gastruloids provided as smoothed curves. Normalized fluorescence was plotted over the anterior-posterior (A-P) axis normalized distance. FIG. 5C left: Immunofluorescence Z-slices of FOXA2 (cyan)/Ki67 (magenta) depicts primitive foregut (fg) relative to cardiogenic region in day 5 versus day 6 EMLOC gastruloids. Right: CDH1/E-Cadherin depicts primitive gut tube (red) positioning relative to cTnT+ cardiogenic region (magenta). Cells are counterstained with DAPI. FIG. 5D immunofluorescence Z-slices of GATA6 (cyan)/type 1 collagen (Col1, magenta) depicts primitive gut tube relative to cardiogenic region during chamber precursor formation in day 5 (left) versus day 6 (right) EMLOC gastruloids. FIG. 5E cTnT (magenta) and CDH2/N-Cadherin (cyan) co-localization in cardiac crescent reveals epithelization of cardiomyocyte progenitors. Inset depicts cTnT+ sarcomeres without pseudocolor. High magnification images with and without DAPI are provided (right) at the boundary of the EMLOC cardiogenic region. Individual scale bars provided.

FIG. 6. Myocardial expansion and heart tube morphogenetic specialization. FIG. 6A cartoon schematic of cardiogenesis from cardiac crescent to heart tube specialization and heart tube looping. Truncus arteriosus (TA), sinus venosus (SV). FIG. 6B heart tube staging in day 7 EMLOC gastruloids by cTnT (magenta) and TUJ1 (red), corresponding to the boxed stage in FIG. 6A. Maximally projected Z-stacks and single Z-slice is shown. FIG. 6C 3D reconstruction of the anterior cardiac region with cTnT (magenta) and DAPI (grey) depicting putative outflow tract (yellow arrows) and chambers in two EMLOC gastruloids. FIG. 6D multi-dimensional visualization of cTnT (magenta) and DAPI (grey). Sagittal and transverse planes are shown. Individual scale bars provided. Asterisks (*) indicate communication with proximal EMLOC compartment. Individual scale bars provided.

FIG. 7. Chamber precursor morphogenesis in EMLOCs. FIG. 7A cartoon schematic of a developing heart tube. Chamber wall layers are expanded to depict myocardium, extracellular matrix-rich cardiac jelly, and endocardium interior lining. FIG. 7B 3D reconstruction of the anterior cardiac chamber-like structures with cTnT+ myocardium (magenta) and Laminin+ interior (top-left). Single Z-slice multi-dimensional view of chamber (top-right). High magnification images are provided below. FIG. 7C VCAN, ADAMTS1, ANGPT1 genes involved in cardiac jelly and its spatiotemporal degradation in day 16 EMLOC by scRNAseq, visualized using PHATE. FIG. 7D immunofluorescence of cTnT, GATA4 and DAPI demonstrating putative outflow tract (white arrows) in two day 7 EMLOCs. Z-slice inset provides a second example. FIG. 7E immunofluorescence of N-Cadherin (CDH2, cyan), VE-Cadherin (vascular endothelial cadherin/CD144/CDH5, magenta), and DAPI (grey) depicting endothelial biomarker expression lining the putative outflow tract. Rightmost zoom image depicts valve-like crescent structures. Individual scale bars provided. FIG. 7F biomarkers for smooth muscle cells (CNN1/TAGLN) and the outflow tract (ISL1/PDE5A/CDH11) along with vascular endothelial cells (KDR/FLT1/ESAM/CDH5) (cluster 9). FIG. 7G POSTN, TBX3, NPR3, NFATC4 genes involved in atrioventricular valve formation.

FIG. 8. Neuron co-development and population of the cardiac region. FIG. 8A neural rosette biomarkers SOX2 (cyan) and TUJ1 (red) emerging posteriorly in day 7 H3.1.1 EMLOC, counterstained with DAPI. FIG. 8B and FIG. 8C SOX2+ rosette and neurogenesis (TUJ1) counterstained with DAPI along with high magnification FIG. 8C. Inset is whole EMLOC. FIG. 8D immunofluorescence of cTnT (cyan) and TUJ1 (red) depicting single neuron in day 8 EMLOC (left, white dotted box) and zoom in (middle panel). Comparison with TUJ1+ neuron tract in day 18 EMLOC (far right panel). FIG. 8E Absolute number of TUJ1+ cells in day 7 versus day 18 EMLOCs (n=10 per time point). ****p<0.0001, t=8.929, df=18 by unpaired two-tailed t-test. FIG. 8F proportion of EMLOCs with TUJ1+ neuronal fibers distributed within the cardiac region at day 7 versus day 18 (N=4 replicate experiments; day 7: 3.3+/−1.9% of population; day 25: 55.8+/−2.9; **p=0.0011, t=12.58, df=3 by paired two-tailed t-test). FIG. 8G nidus of neurogenesis posterior to gut tube endodermal cells and cardiogenic region in day 8 EMLOC by TUJ1 (red), GATA6 (cyan), and cTnT (magenta). Maximally projected Z-stack (Z-total) and single Z-slice shown. White arrows point to communicating channels. Gut tube endoderm is laminated while neural rosettes are continuous with surrounding GATA6+ cells. FIG. 8H left: UMAP representation of day 16 EMLOC scRNAseq highlighting trunk neural progenitors (ZIC1/RFX4), sympathetic neurogenesis (INSM1/ISL1) and Schwann cell glia (SOX10/PLP1). Right: quantification of neuronal class proportions in clusters 1, 8, 10 as autonomic (ASCL1 62%, PHOX2B 9%), sensory (POU4F1 25%) and motor (MNX1 4%). FIG. 8I genes in the cardiogenic region involved in neuronal patterning and innervation. NPY, BDNF, SEMA3A, PRPH, EDNRA, ISL1 shown by UMAP (day 16). FIG. 8J phase contrast image of contractile EMLOC chamber-like structures. FIG. 8K 3D reconstruction of TUJ1+ neuronal fiber intercalation with chamber-like myocardium in day 25 EMLOC. Rotated view is shown. Individual scale bars provided.

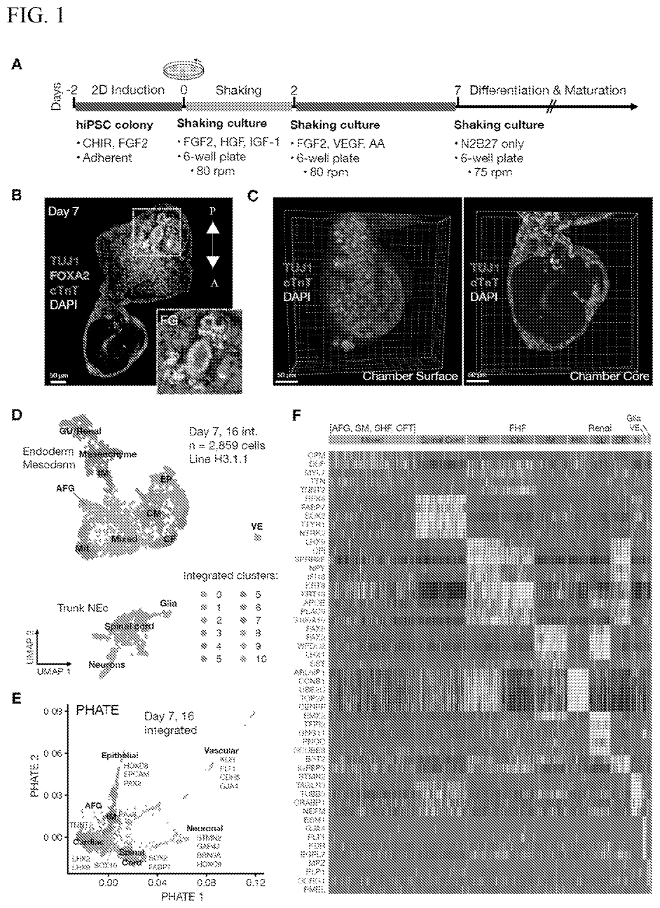

FIG. S1. Directed developmental cardiogenesis and spontaneous contractility in modified neuro-gastro-cardiac EMLOs (EMLOCs), related to FIG. 1A. FIG. S1A overview of protocol for EMLOC gastruloid generation. Cardiogenesis was induced at 48 h post-aggregation by addition of VEGF and ascorbic acid (AA). FIG. S1B phase contrast image of 2D 0 colonies 24 h after induction with N2B27+CHIR/FGF2. FIG. S1C EMLOC Suspension cultures 24 h and 48 h after dissociation and spontaneous aggregation on the orbital shaker. FIG. S1D Day 1 aggregates do not express GATA6 (yellow) and exhibit non-uniform FOXA2 expression (cyan) by immunofluorescence. FIG. S1E immunofluorescence in a single day 1 aggregate (white dotted line) demonstrates uniform, non-polarized expression of SOX2 (magenta). Cells are counterstained with DAPI (grey). FIG. S1F phase contrast images of polarized gastruloids with spontaneously contracting cardiogenic chambers (day 7). Annotated EMLOC is shown (right). FIG. S1G two immunofluorescence Z-slices with cardiac biomarkers GATA6 (blue) and cardiac Troponin-T (cTnT, magenta) in a day 7 gastruloid demonstrating chamber cytoarchitecture. Images are representative of the general EMLOC population at these stages. Individual scale bars provided. FIG. S2. UMAP and PHATE visualization of scRNAseq for day 7 and day 16 EMLOC time points, related to FIG. 1. (A) UMAP representation of day 7 (1,004 cells) and day 16 (1,855 cells) according to the integrated dataset with ten clusters. FIG. S2B PHATE representation of samples in FIG. S2A.

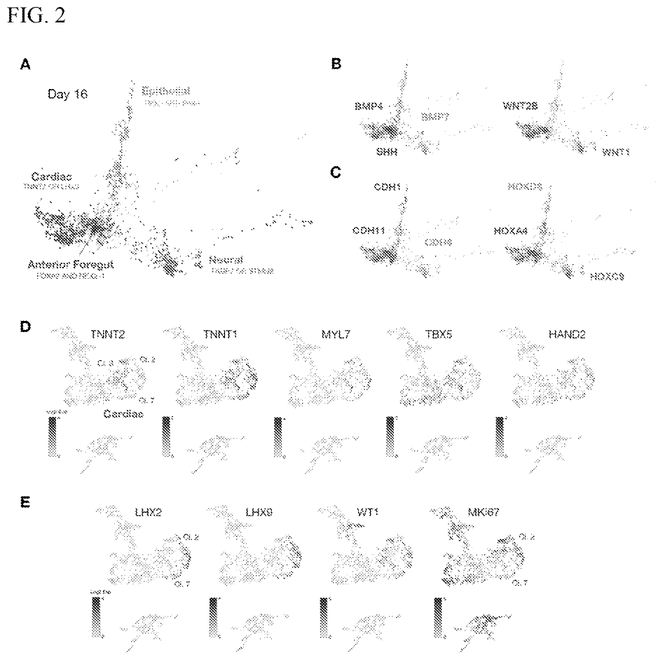

FIG. S3. Developmental features of cardiogenesis in EMLOCs, related to FIG. 2. FIG. S3A characteristic gene biomarkers for splanchnic mesoderm (FOXF1, PDGFRA, TWIST1, PRRX2). FIG. S3B gene biomarkers of ventricular cell specification and morphogenesis (GATA4, GATA6, CDH2, NFIA, ISL1, FAT, ALDH1A2, TTN). FIG. S3C PHATE (left) and UMAP (right) visualization of cells co-expressing ETS1, EDNRA, TGIF1, HOXA2 consistent with a cardiac neural crest cell phenotype.

FIG. S4. EMLOCs generate the appropriate cardiac ECM milieu, related to FIG. 2. FIG. S4A four collagen genes (COL1A1, COL1A2, COL3A1, COL6A3). FIG. S4B glycoprotein (FN1) and proteoglycan (DCN) genes along with EMILIN1, PLAC9. FIG. S4C three laminin genes (LAMA1, LAMA4, LAMA5) show distinct distributions by PHATE. Asterisk (*) indicates region of highest expression (Cui et al., 2019).

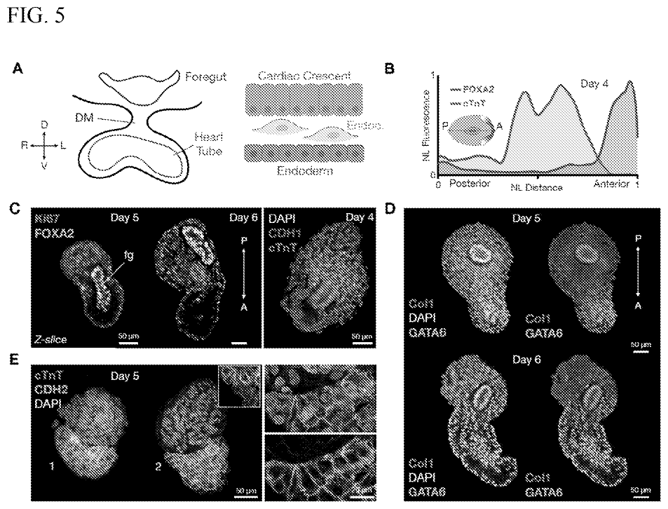

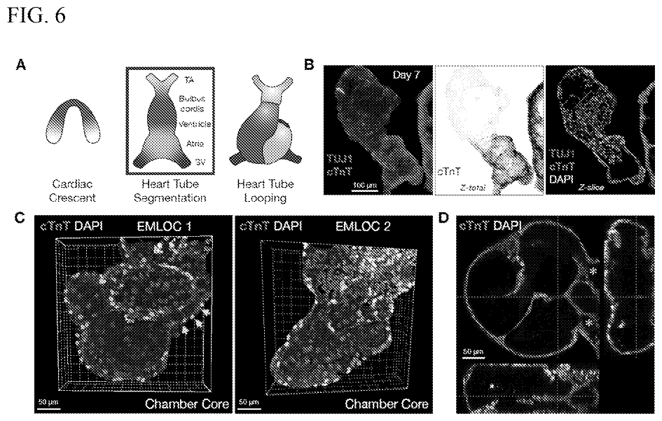

FIG. S5. Assessment of cardiomyocyte morphology with differentiation and morphogenesis states, related to FIG. 5. FIG. S5A relative proportions of day 7 EMLOCs with rounded, proliferating cardiomyocyte progenitors versus flattened, mosaic cardiomyocyte progenitors suggests co-existing phases of differentiation and morphogenesis as previously described for mouse cardiogenesis (N=4 replicate experiments, n.s. p=0.1776, t=1.754, df=3 by paired two-tailed t-test). FIG. S5B immunofluorescence examples of rounded, proliferating progenitors (top: cTnT, Ki67) versus flat, mosaic progenitors (bottom: cTnT, CDH2, DAPI). Left inset is example of whole EMLOC with rounded cardiomyocytes. Individual scale bars provided.

FIG. S6. Additional characterization of the gut tube in EMLOCs, related to FIG. 5. FIG. S6A day 4 EMLOCs with visible gut tube endoderm. FIG. S5B FOXA2+/CDH1+ gut tube endoderm is self-organized posterior to the GATA4+ cardiac crescent. White arrow points to early serous lining of the cardiac crescent. FIG. S5C FOXA2+ gut tube endoderm is laminated, where has TUJ1+/GATA4− neural rosettes are more continuous with surrounding cells, labeled in FIG. S5D. TUJ1 was also observed in mitotic spindle MTs (white arrow). Individual scale bars provided.

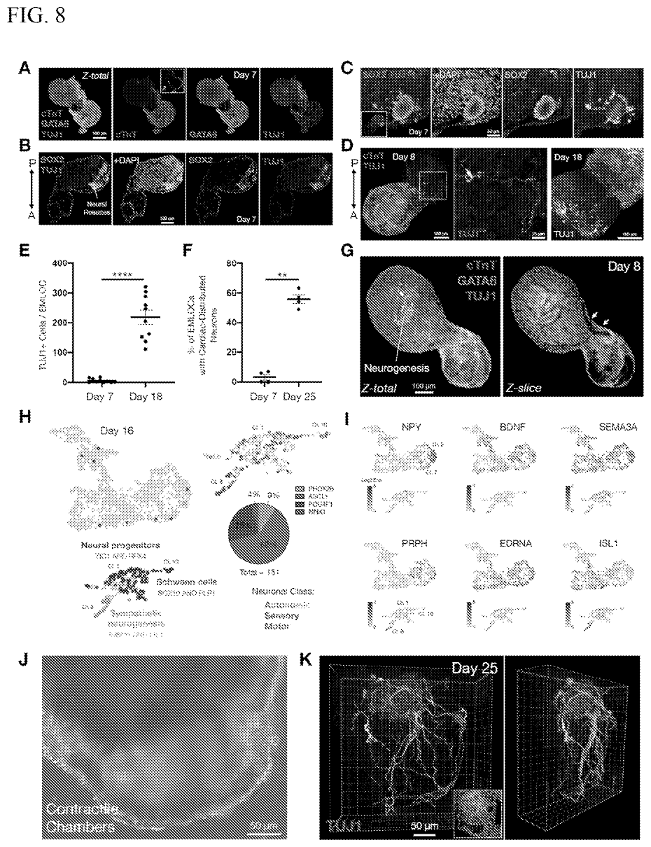

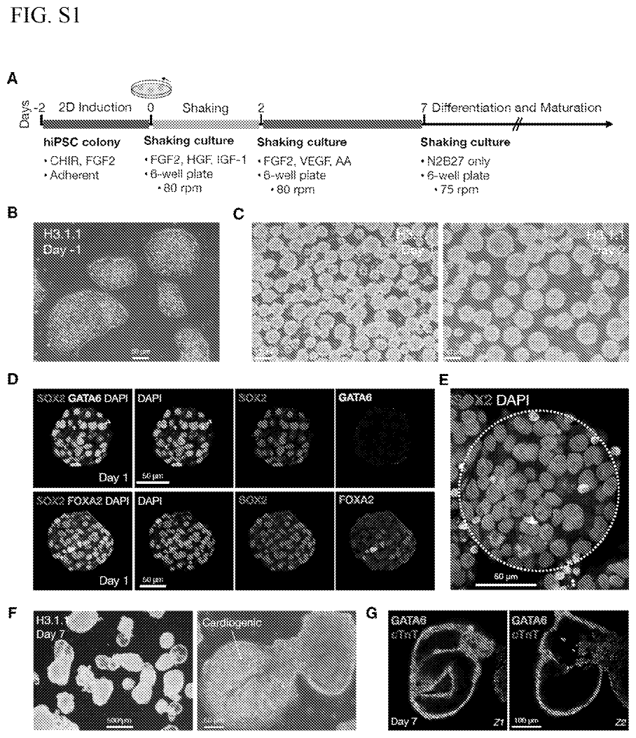

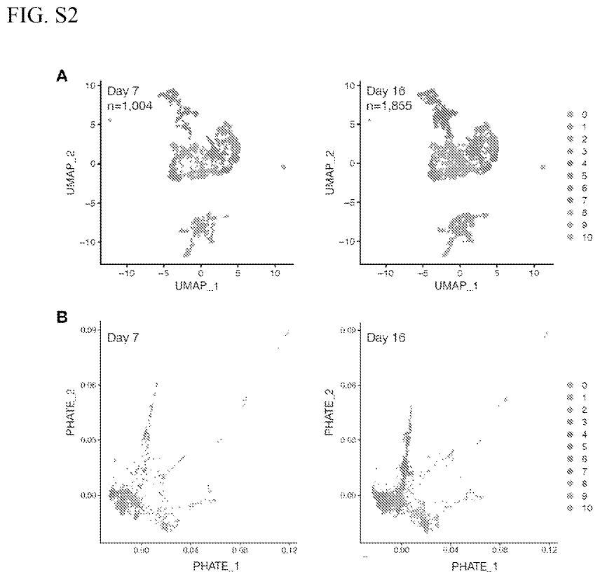

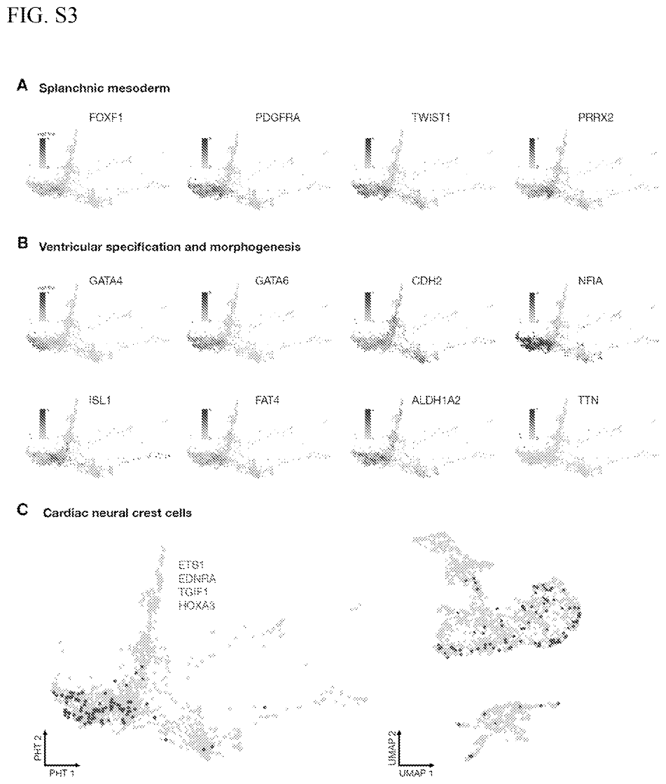

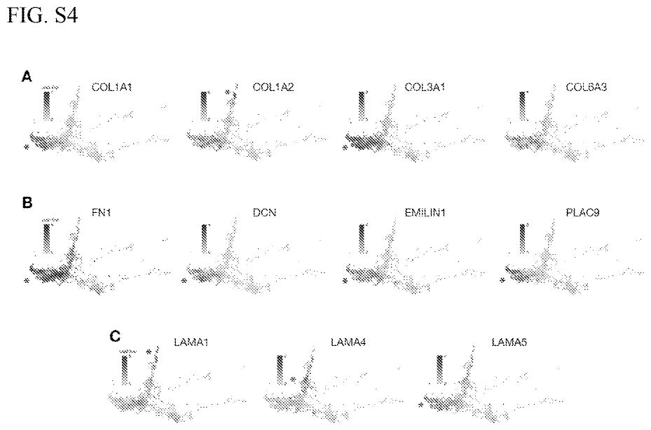

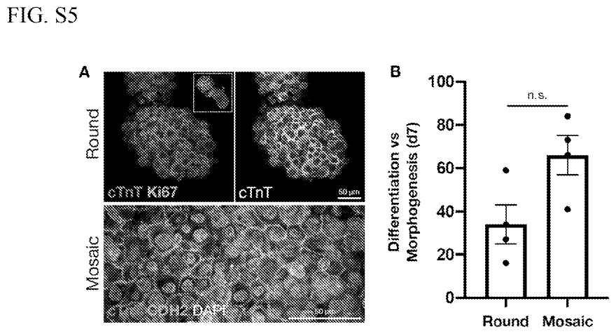

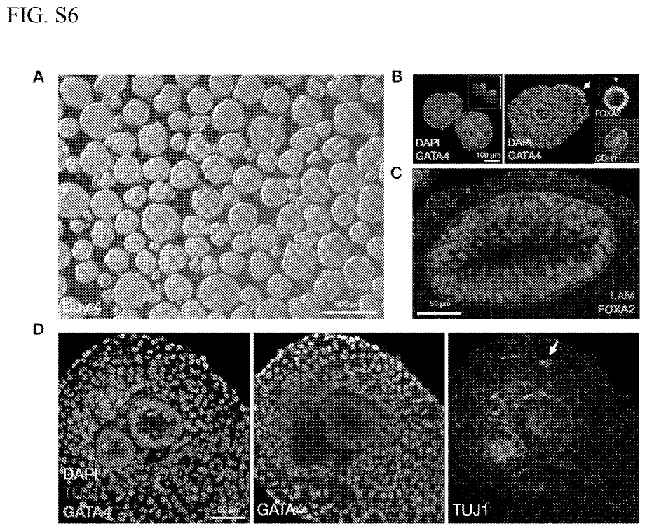

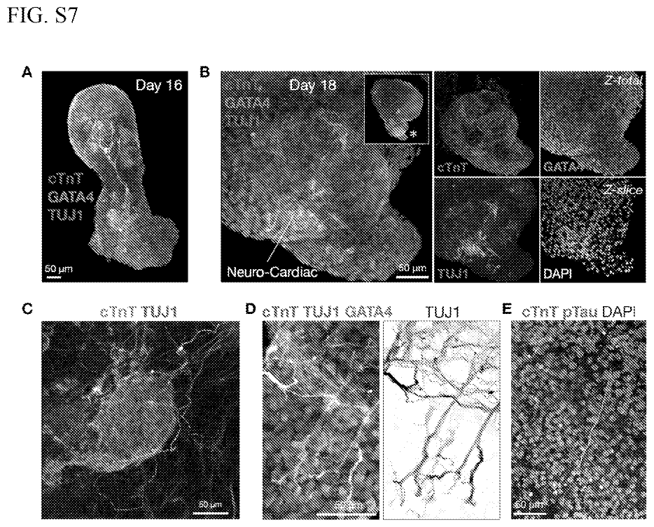

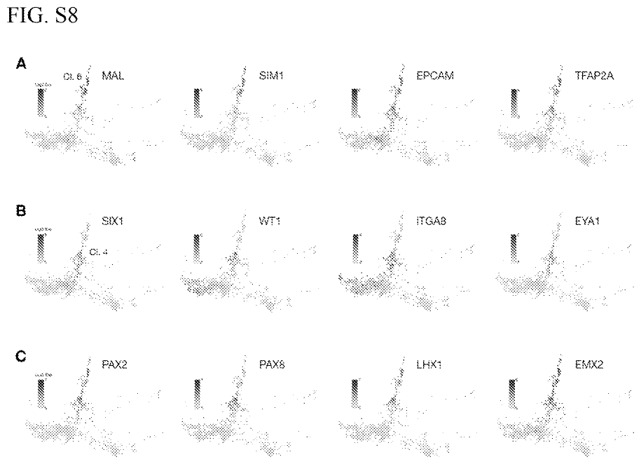

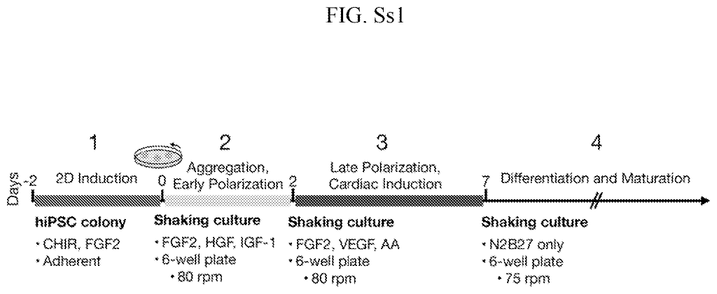

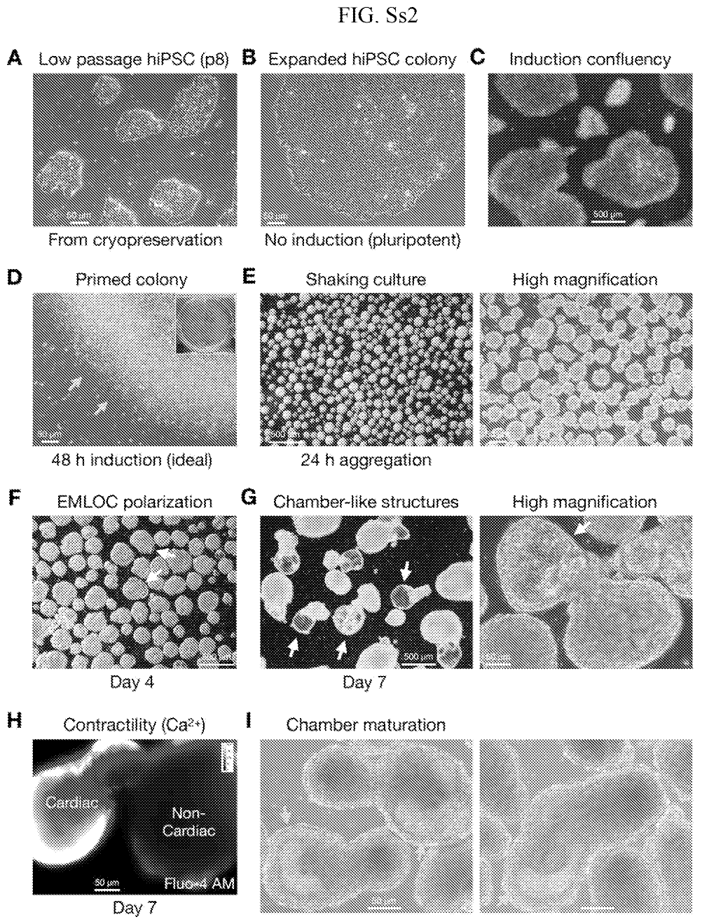

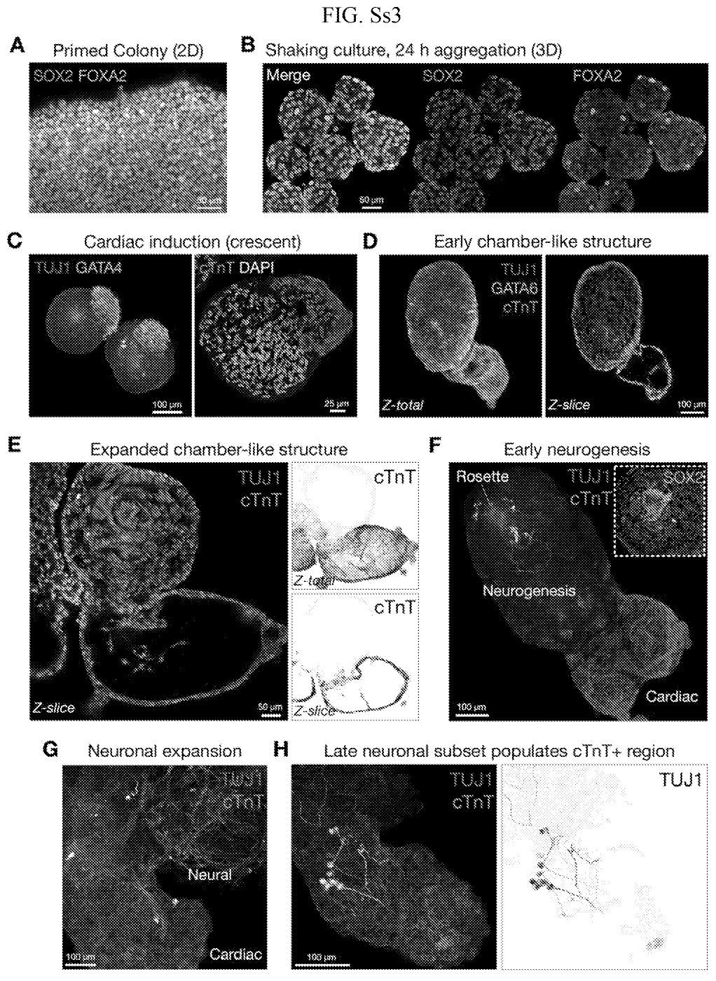

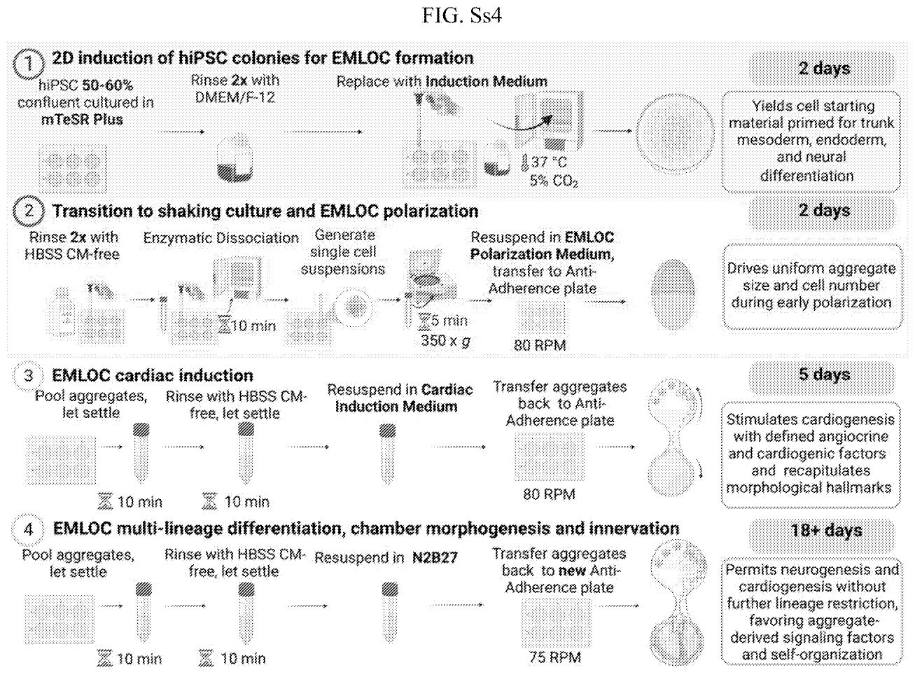

FIG. S7. Additional characterization of neuronal fibers in EMLOCs, related to FIG. 8. FIG. S7A Immunofluorescence of cardiac biomarkers cTnT (magenta) and GATA4 (cyan) along with TUJ1 (red) in day 16 H3.1.1 EMLOCs demonstrate the emergence of neurons proximal to the cardiac region. FIG. S7B immunofluorescence of cTnT (magenta) and TUJ1 (red) at day 18 depicts ganglionated neuronal plexuses in cardiogenic region. Merge is shown with individual cTnT, GATA4 (cyan), TUJ1 channels. Left inset is low magnification image of EMLOC. FIG. S7C TUJ1+ neuronal fiber termination (red) onto cTnT+ cells (cyan) reminiscent of nodal innervation. FIG. S7D Cardiac biomarkers cTnT (magenta) and GATA6 (cyan) with TUJ1. FIG. S7E axons identifiable by pTau within the cTnT+ myocardium (right). Individual scale bars provided.

FIG. S8A-S8C shows additional gene profiles of the present disclosure.

FIG. Ss1 and FIG. Ss4 describe the generation of EMLOCs from the hiPSC line H3.1.1 derived from fibroblasts of a self-designated Hispanic-Latino donor.

FIG. Ss2A presents low passage hiPSC, FIG. Ss2B presents expanded hiPSC colony, FIG. Ss2C presents induction confluency, FIG. Ss2D presents primed colony, FIG. Ss2E presents shaking culture, FIG. Ss2F presents EMLOC polarization, FIG. Ss2G presents chamber-like structures, FIG. Ss2H presents contractility, and FIG. Ss2I presents chamber maturation.

FIG. Ss3A presents primed colony (2D), FIG. Ss3B presents shacking culture 24 h aggregation (3D), FIG. Ss3C presents cardiac induction (crescent), FIG. Ss3D presents early chamber-like structure, FIG. Ss3E presents expanded chamber-like structure, FIG. Ss3F presents early neurogenesis, FIG. Ss3G presents neural expansion, and FIG. Ss3H presents late neural subset populates cTnT+region.

### DETAILED DESCRIPTION

The present disclosure is directed towards compositions, kits, and methods of extending/refining elongating multi-lineage organized (EMLO) gastruloids to cardiac (EMLOC) gastruloids, and the generation of interconnected neuro-cardiac lineages in a single gastruloid model.

Stem cell technology enables unprecedented studies of human multi-lineage development through the ability to recapitulate embryonic and post-embryonic dynamic regulatory processes in a multicellular context. As such, it is expected to accelerate ex vivo strategies in organ development. Reproducing human cardiogenesis remains challenging, requiring spatiotemporal paracrine inductive and mechanical cues by multi-lineage tissues. Here, elongating multi-lineage organized (EMLO) gastruloids has been extended to cardiac (EMLOC) and the present disclosure describes the generation of interconnected neuro-cardiac lineages in a single gastruloid model. In embodiments, the contractile EMLOCs of the present disclosure recapitulate numerous interlinked developmental features of heart tube formation and specialization, cardiomyocyte differentiation and remodeling phases, epicardium, ventricular wall morphogenesis, and formation of a putative outflow tract. Along with cardiogenesis in EMLOCs that originates anterior to the gut tube primordium, neurons were observed that progressively populate the cardiogenic region in a pattern that mirrors spatial distribution of neurons in heart innervation. In embodiments, the EMLOC model represents an important multi-lineage advancement for the study of human cardiogenesis with co-developed neuronal integration.

The present EMLOC gastruloid technology arises through directed morphogenic cellular changes. Three-dimensional cell aggregates progress from spherical to an ovoid shape that develop into an elongated structural form. During morphogenesis changes to the cells are co-directed to neural and cardiac cell lineages that are spatially compartmentalized. This polar state is recognized by cell lineage specific biomarkers and distinct cardiac-morphogenic events. Integration of the cell lineages occurs when neuronal progenitors migrate into the cardiac region to organize neurogenesis of the heart tissue. This elongation of cell structure is not seen in other cardiac organ models.

In embodiments, EMLOs are coaxed or caused developmentally, by providing the necessary cues, towards more extended cardiac differentiation with reproducible morphogenesis. In embodiments, EMLO formation techniques are altered to include angiocrine and pro-cardiogenic factors such as factors previously detailed (Rossi et al., 2021). The present disclosure now provides a gastruloid strategy for neuro-cardiac co-developed tissues that recapitulate aspects of early human heart morphogenesis with neuronal integration. In embodiments, multiple events are tracked in cardiomyocyte differentiation from splanchnic mesoderm and spontaneous contractility, and chamber precursor formation was observed along with early constrictions and septations, epicardium, and putative structures resembling the outflow tracts. Importantly, EMLOC embodiments, of the present disclosure not only retain the interacting neural compartment but achieve neurogenesis to generate an organized co-developed neuro-cardiac gastruloid. It has now been demonstrated that EMLOCs are suitable for use as an advanced model for human cardiogenesis and the integration with endoderm and neurons towards the goal of organ innervation.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [Cl 95%] for the mean) or within ±10% of the indicated value, whichever is greater.

As used herein the terms “under culture conditions” or “under culturing conditions,” as known in art, for example, includes plastic dished in CO2 chambers to culture cells.

As used herein the term “contractile innervated tissue,” refers to the multichambered heart that has neurons integrated from the neural region of the EMLOC over the heart chambers and the ability of the whole heart organ to contract in beats.

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

As used herein the term “effective amount” as used herein means that amount of an agent that elicits the biological or medicinal response in a cell, tissue, organ, system, animal, or human that is being sought by a researcher, veterinarian, medical doctor, or other clinician. In some embodiments, the effective amount is a “therapeutically effective amount” for the alleviation of the symptoms of the disease or condition being treated. In some embodiments, the effective amount is a “prophylactically effective amount” for prophylaxis of the symptom s of the disease or condition being prevented.

The term “elongating multi-lineage organized cardiac gastruloids,” or “EMLOC gastruloids,” refers to a multi-step process of morphological and cell lineage differentiation steps that mimics normal human heart development. The morphological changes of the cardiac gastruloids encompass various forms as they develop. The cardiac gastruloid forms include, but are not limited to, round to ovoid to tube-like and/or hour-glass shapes. These forms/shapes coincide with heart development.

The term “trunk biased” refers to the exclusion of anterior neural tissue/structures, and the term “cardiac permissive” refers to the atypical aspect of the presently disclosed protocol that allows for the co-generation of both cardiac and neural cell types without restriction to one or the other. Cardiac permissive directed generation within a domain of progenitor cells necessary for cardiac morphogenesis.

The term Trunk-biased stem cells refers to the discovery of distinct lineages in mammals, including humans for stem cells of the body trunk that can be mimicked in vitro, and excluding anterior structures such as brain and brainstem.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

In embodiments, the present disclosure relates to EMLOC kits, EMLOC compositions, or one or more methods of making EMLOCs.

In embodiments, the present disclosure includes a method of generating elongating multi-lineage organized cardiac (EMLOC) gastruloids, including: contacting a colony of human induced pluripotent stem cells with hepatocyte growth factor (HGF), insulin-like growth factor-1 (IGF-1), fibroblast growth factor-2 (FGF-2) under culture conditions for a first duration to form a first plurality of gastruloids; and contacting the first plurality of gastruloids with vascular endothelial growth factor (VEGF), fibroblast growth factor-2 (FGF-2), and ascorbic acid under culture conditions for a second duration to form EMLOC gastruloids. In embodiments, the growth factor (HGF), insulin-like growth factor (IGF), fibroblast growth factor (FGF) are provided in an effective amount to coax/stimulate/induce the colony of human induced pluripotent stem cells to form a first plurality of gastruloids. In embodiments, the vascular endothelial growth factor (VEGF), fibroblast growth factor (FGF), and ascorbic acid are provided in an effective amount to coax the first plurality of gastruloids to form EMLOC gastruloids. In embodiments, the human induced pluripotent stem cells are contacted under culture conditions with 2 ng/ml hepatocyte growth factor (HGF), 2 ng/ml insulin-like growth factor (IGF), 10 ng/ml fibroblast growth factor (FGF) for a first duration, and 5 ng/ml vascular endothelial growth factor (VEGF), 30 ng/ml fibroblast growth factor (FGF), and 0.5 mM ascorbic acid for a second duration. In embodiments, the contacting occurs in an environment characterized as cardiac permissive. In embodiments, the colony of human induced pluripotent stem cells is characterized as trunk-biased stem cells. In embodiments, the first duration is 24 hours, and the second duration is 2-7 days. In embodiments, the colony of human induced pluripotent stem cells are trunk-biased stem cells.

In some embodiments, the present disclosure relates to one or more EMLOC gastruloids formed by one or more methods of the present disclosure. In embodiments, the present disclosure includes one or more EMLOC gastruloids, including a three-dimensional structure including or consisting of contractile innervated tissue.

In embodiments, the present disclosure includes a method of generating a contractile innervated human heart tissue or organ, including: coaxing or promoting an exogenous population of stem cells to divide and differentiate to an innervated cardiac fate by contacting the exogenous population of stem cells with hepatocyte growth factor (HGF), insulin-like growth factor-1 (IGF-1), fibroblast growth factor-2 (FGF-2) under culture conditions for a first duration to form a first plurality of gastruloids; and contacting the first plurality of gastruloids with vascular endothelial growth factor (VEGF), fibroblast growth factor-2 (FGF-2), and ascorbic acid under culture conditions for a second duration.

In embodiments, the present disclosure includes a method of generating an organized co-developed neuro-cardiac gastruloid, including: contacting an exogenous population of stem cells with an effective amount of hepatocyte growth factor (HGF), insulin-like growth factor (IGF-1), fibroblast growth factor (FGF-2) for a first duration, and subsequently contacting the exogenous population of stem cells with an effective amount of vascular endothelial growth factor (VEGF), fibroblast growth factor (FGF-2), and ascorbic acid for a second duration. In embodiments, the exogenous population of stem cells are characterized as adherent and/or induced by contacting the cells with a biologically active stem cell differentiation and reprogramming reagent and a fibroblast growth factor 2 (FGF-2).

In embodiments, the present disclosure includes hiPSC technology for formation of a contractile innervated human heart from a three-dimensional microenvironment. In embodiments, the present disclosure establishes the 3D microenvironment and developmental regulators and signaling molecules necessary to recapitulate human embryonic temporal events in heart formation. In embodiments, the technology enables three-dimensional innervation studies to non-neural tissue and organs in an embryo-like model. The technology has broad applications to provide fundamental insights into congenital heart disease, cardiac pathophysiology, cardiac hypoxia, and pharmacotherapy relevant to neuromodulation of cardiomyocytes, neurocardiogenic syncope, and other neural-based arrhythmia pathologies for embryonic to adult biomedical analysis. This technology further has biomedical relevance for neuro-gastro-cardiac development.

In some embodiments the present disclosure relates to gene expression from a single cell RNA sequence and data related thereto.

In embodiments, EMLOCs of the present disclosure mirror in vivo development of distinct cardiac, neural and epithelial lineages including primitive foregut, in signaling networks, adhesion proteins and transcription factors.

In embodiments, EMLOCs of the present disclosure generate trunk and neuroectoderm/spinal cord progenitors, trunk neurons, peripheral glia/Schwann cells.

In embodiments, EMLOCs of the present disclosure recapitulate numerous key features of human cardiogenesis including cardiac crescent transformation into the contractile heart tube, cardiomyocyte differentiation versus remodeling phases, and formation of chamber- and outflow tract-like structures. Cardiogenesis occurs anterior to primitive gut tube-like endodermal cells that in vivo are thought to be required.

In embodiments, EMLOCs of the present disclosure generate intermediate mesoderm, metanephric mesenchyme, genitourinary/renal epithelium.

In embodiments, EMLOCs of the present disclosure retain splanchnic mesoderm biomarkers: GATA4, GATA6, FOXF1, PDGFRA, TWIST1, and PRRX2.

In embodiments, EMLOCs of the present disclosure generate diverse cell types arising from splanchnic mesoderm, including cardiomyocytes that form myocardium, cells of the proepicardium and epicardium, and cardiac fibroblasts.

In embodiments, EMLOCs of the present disclosure generate cardiomyocytes, epicardial cells and cardiac fibroblasts and vascular endothelium.

In embodiments, EMLOCs of the present disclosure generate primarily ventricular cardiomyocytes at day 16 timepoint.

In embodiments, EMLOCs of the present disclosure express first heart field (FHF) and second heart field (SHF) genes, including: TBX5 and HAND2 (both) and NKX2-5, HAND1 (FHF) and MEF2C, ISL1, TBX18 (SHF).

In embodiments, EMLOCs of the present disclosure express genes for regulating ventricle growth and morphogenesis.

In embodiments, EMLOCs of the present disclosure express fetal heart development genes, including: KRT8/KRT18, APOE, PLAC9 and S100A10.

In embodiments, EMLOCs of the present disclosure express epicardial genes including: WT1, TCF21, TPJ1, LHX2, LHX9, TBX18, PLAC9.

In embodiments, EMLOCs of the present disclosure express upregulated cardiac fibroblast genes, including IGFBP5, a biomarker associated with cardiac fibroblast activation and BTS2, and/or a biomarker of mature cardiac fibroblasts.

In embodiments, EMLOCs of the present disclosure express cardiomyocyte and fibroblast-derived ECM genes, including biomarkers of cardiac jelly ECM and its spatiotemporal degradation by day 16 (VCAN, ADAMTS1, ANGPT1).

In embodiments, EMLOCs provide for the presence of cardiogenic mesoderm arising cells, including: contractile cells (TNNT1-slow type troponin, TNNT2-cardiac troponin, MYL7), outflow tract cells (PDE5A, ISL1, FN1, MEGF6, MSX2, SEMA3C, EMILN1, CNN1, TAGLN) and atrioventricular conduction and organization (GJA1, CACNA1H, TBX3, NRP2, CXCL12, DSP).

In embodiments, by comparing calcium transients per minute in EMLOC system versus the mESC cardiogenesis study in gastruloids, a similar species-specific ratio to that between the resting adult heart rate in human versus mouse (˜8× higher in the murine model) that have similar processes of cardiogenesis was observed or provided.

In embodiments, EMLOCs of the present disclosure express anterior foregut progenitor cells (HHEX, SHH, FOXA2) and anterior foregut identity (FOXA2, NKX2-1, SHH, EPCAM).

In embodiments, EMLOCs of the present disclosure express cardiac neural crest cell biomarkers (ETS1, EDNRA, TGIF1, HOXA3).

In embodiments, EMLOCs of the present disclosure express genes involved in left-right asymmetry specification during in vivo cardiogenesis by day 16 (IRX3, HAND1, PITX2, RTTN).

In embodiments, EMLOCs of the present disclosure express genes that are biomarkers for smooth muscle (CNN1/TAGLN), outflow tract development (ISL1/PDE5A/CDH11) and well-differentiated vascular endothelium (KDR/FLT1/ESAM/CDH5), and Nodal and valvar biomarkers (POSTN/TBX3/NPR3/NFATC4).

In embodiments, EMLOCs of the present disclosure display an emergence of neurons at day 7 and neuronal expansion by day 16 (>40 fold) that includes neural progenitors (ZIC1, RFX4, HES5, FABP7, EDNRB, NTRK2, OLIG3, MSX1) and specialized neuronal subtypes (INSM1, ELAVL3, DLG4, CAMK2A, SLC18A3, SLC17A6, CHRNA3, NTRK3).

In embodiments, EMLOCs of the present disclosure express, by day 16 neural crest-derived Schwann cells (SOX10, PLP1, MPZ, S100B, TFAP2B, NGFR).

In embodiments, EMLOCs of the present disclosure contains by day 16 combined INSM1/ISL1 expression that indicates that sympathetic neurogenesis occurs in EMLOCs, which is particularly relevant to developing cardiac innervation. Restricted expression of HOXC6 and HOXC9 to these clusters supports spinal cord and trunk identity.

In embodiments, EMLOCs of the present disclosure contain biomarkers for specialized neuronal subtypes of autonomic neurons that is ASCL1 (93/151 neurons, ˜62%) and PHOX2B (14/151 neurons, ˜9%) that predominated versus sensory neurons POU4F1/BRN3A (38/151 neurons, ˜25%) or motor neurons MNX1/HB9 (6/151 neurons, ˜4%). This finding is distinct from EMLOs (Olmsted and Paluh, 2021a), in which motor neurons were primarily generated.

In embodiments, EMLOCs of the present disclosure express biomarkers of cardiac innervation: neuropeptide Y (NPY), brain-derived neurotrophic factor (BDNF), semaphorin 3A (SEMA3A), peripherin (PRPH), endothelin receptor type A (EDNRA), and ISL1. Genes involved in autonomic neurogenesis and cardiogenesis such as ISL1 also play a role in development and innervation of cardiac pacemaker cells that dictate automaticity and participate in the conduction system apparatus.

In embodiments, EMLOCs of the present disclosure express neuronal fiber patterning on cardiomyocytes and reflect normal developmental patterning and ability to respond to intrinsic spatial cues.

In embodiments, EMLOCs of the present disclosure show terminating neuronal fibers on cardiomyocytes identified by phosphor-tau (Ser214) immunostain.

In embodiments, EMLOCs of the present disclosure are formed in a cardiac permissive microenvironment created by two-day treatment of trunk-biased stem cells aggregated by orbital shaking in suspension with factors added in starting concentrations of: 2 ng/ml HGF, 2 ng/ml IGF-1, 10 ng/ml FGF-2 and switching to 5 ng/ml VEGF, 30 ng/ml FGF-2, 0.5 mM ascorbic acid from day 2 to day 7.

In embodiments, elongating multi-lineage organized (EMLO) gastruloids can act as trunk-biased, cardiac-permissive starting material.

In embodiments, methods of the present disclosure provide a rapid timeline of 3D cardiogenesis from beating cardiomyocytes at day 7 to innervated cardiac region by day 25.

In embodiments, methods of the present disclosure provide neuronal co-emergence by day 7 that are repelled from the cardiogenic region.

In embodiments, methods of the present disclosure provide for timing of neuronal interaction with reduced cardiac jelly extracellular matrix between day 7 and day 16 to populate the cardiogenic region.

In embodiments, methods of the present disclosure provide EMLOCs with innervation mirroring human embryonic heart patterning by day 25.

In embodiments, methods of the present disclosure provide EMLOCs with cardiac crescent formation from day 4 to day 6.

In embodiments, methods of the present disclosure provide EMLOCs with progression of GATA4+/GATA6+ expressing cardiogenic region into a cTnT+ co-expressing region indicative of cardiomyocyte formation.

In embodiments, methods of the present disclosure provide EMLOCs with embryonic-similar (human weeks 3-5) heart tube formation and cell type specialization.

In embodiments, methods of the present disclosure provide EMLOCs with cardiomyocyte transitions between flat or rounded cardiomyocyte cell shapes consistent with alternating phases of cardiomyocyte morphogenesis and differentiation in ventricular wall morphogenesis.

In embodiments, methods of the present disclosure provide EMLOCs with formation of communicating cardiac chambers with embryonic-similar organization (myocardium, cardiac jelly, endocardium).

In embodiments, methods of the present disclosure provide EMLOCs with formation of CD144 endocardium interior chamber lining and valve-like outflow tract.

In embodiments, methods of the present disclosure provide EMLOCs with cardiomyocyte contractility with innervation in 3D heart model.

In embodiments, methods of the present disclosure provide EMLOCs with contractility relative to human beats/s (median ˜10 calcium transients per minute, range 5-20 calcium transients per minute).

In embodiments, methods of the present disclosure provide EMLOCs with formation of a channel positional to the cardiac chamber outflow tract.

In embodiments, methods of the present disclosure provide EMLOCs with an ability to switch from growth factor-applied to growth factor-free medium after contractility begins at day 7.

In embodiments, methods of the present disclosure provide EMLOCs with neuron-cardiomyocyte synapse formation in a 3D heart mode.

In embodiments, methods of the present disclosure provide EMLOCs with cardiogenesis anterior to formation of gut tube primordium in a 3D heart model.

In some embodiments, the present disclosure includes a method of preparing heart-like tissue, including: contacting a colony of human induced pluripotent stem cells with hepatocyte growth factor (HGF), insulin-like growth factor-1 (IGF-1), fibroblast growth factor-2 (FGF-2) under culture conditions for a first duration; and subsequently contacting the colony of human induced pluripotent stem cells with vascular endothelial growth factor (VEGF), fibroblast growth factor-2 (FGF-2), and ascorbic acid under culture conditions for a second duration to form heart-like tissue. In some embodiments, the heart-like tissue is characterized as including a plurality of EMLOC gastruloids. In embodiments, the colony of human induced pluripotent stem cells are contacted with an effective amount of hepatocyte growth factor (HGF), insulin-like growth factor-1 (IGF-1), fibroblast growth factor-2 (FGF-2), and combinations thereof. In embodiments, the colony of human induced pluripotent stem cells are contacted with an effective amount of vascular endothelial growth factor (VEGF), fibroblast growth factor-2 (FGF-2), ascorbic acid, and combinations thereof. In embodiments, a heart-like tissue is formed by the methods of the present disclosure. In some embodiments, a “heart-like” tissue refers to tissue differentiated in vitro (e.g., from a stem cell) that has one or more properties of a heart, such as a human heart. In embodiments, the heart-like tissue is characterized as synthetic or non-naturally occurring. In embodiments, the heart-like tissue is characterized as innervated and contractile.

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, and reducing/oxidizing agents, with art-recognized alternatives and using no more than routine experimentation, are within the scope of the present disclosure.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### Example 1

Experimental Models And Subject Details: Human induced pluripotent stem cells. hiPSC lines were derived from Coriell de-identified human fibroblast samples from consenting donors, including the Hispanic-Latino H3.1.1 line used in this study (Chang et al., 2015; Tomov et al 2016). Line H3.1.1 was reprogrammed with Yamanaka factors by the laboratories of Dr. Paluh and Dr. Jose Cibelli from these donor fibroblasts and comprehensively characterized for pluripotency (immunofluorescence, RT-PCR), G-band karyotype, teratoma formation, multi-lineage differentiation, bulk RNA-Seq, ChIP-Seq, and used in multiple studies from this lab. Recent G-band karyotype validation and pathogen analysis was performed by Cell Line Genetics, Inc. (NY, NY). H3.1.1 hiPSC colonies were cultured in mTeSR Plus supplemented with 1× penicillin-streptomycin (P-S) on hESC-qualified Matrigel (1:100 dilution; Corning) in a humidified incubator at 37° C., 5% CO₂. Cultures were passaged 1:6 in 6-well plates every 4-7 days using Gentle Cell Dissociation Reagent (GCDR, STEMCELL Technologies). Cells were cryopreserved in mFreSR.

Method Detail EMLOC Formation: EMLOs were formed similarly to as previously described (Olmsted and Paluh, 2021a, 2021b) with several important differences detailed as follows. H3.1.1 adherent hiPSC colonies were maintained in mTeSR Plus pluripotency medium as described above. At ˜60% confluency, pluripotency medium was changed to induction medium (N2B27 basal medium supplemented with 3 μM CHIR 99021, 40 ng/ml basic fibroblast growth factor FGF2). N2B27 basal medium: 1:1 DMEM/F-12:Neurobasal Plus medium, 2% (v/v) B27 Plus supplement, 1% (v/v) N2 supplement, 1× GlutaMAX, 1×MEM Non-Essential Amino Acids, 1× P-S. Adherent hiPSC colonies were induced for two days the one exchange of fresh medium at 24 h. On the day of aggregation, cells were dissociated with 1:1 Accutase:HBSS (Ca—Mg free) at 37° C. for 5 min followed by manual trituration with a P-1000 pipette. Six-well plates were pre-treated with Anti-Adherence Rinsing Solution (STEMCELL Technologies) for 5 min incubation at room temperature followed by two rinses with equal volumes of HBSS. Cells were resuspended in N2B27 supplemented with 10 ng/ml FGF2, 2 ng/ml IGF-1, 2 ng/ml HGF (R&D Systems) and 50 μM Y-27632 (Tocris Bioscience). For aggregation, the single cell suspensions were added at a density of 2×10⁶ cells/ml (2 ml per well, 4×10⁶ total cells). Gastruloids were aggregated overnight using an orbital shaker at 75 rpm clockwise in a humidified incubator with 5% CO₂. The next day, one-half volume of medium was replaced with fresh medium N2B27 supplemented with 4 ng/ml IGF-1, 4 ng/ml HGF, 20 ng/ml FGF2 to maintain the same concentration of growth factors in the culture medium after one-half volume addition. At 48 h, the entire volume of medium was replaced with N2B27 basal medium supplemented with 5 ng/ml VEGF, 30 ng/ml FGF2, and 0.5 mM ascorbic acid (Rossi et al., 2021). EMLOCs were induced in this medium to day 5. At day 7, the EMLOCs were maintained in non-supplemented N2B27. For orbital shaking culture, cells were aggregated and induced at 80 rpm. Speed was reduced to 75 rpm on day 7.

EMLOC single-cell dissociation by cold activated protease for scRNAseq. Type here (UB): Day 7 and Day 16 EMLOCs were dissociated on their respective time points in differentiation according to a previously protocol described (Olmsted and Paluh, 2021a). In brief, ˜25 EMLOCs from each time point were pooled in a 2 ml centrifuge tube and exposed to 1 ml dissociation solution composed of 10 mg/ml Bacillus licheniformis protease and 125 U/ml DNase in ice-cold 1×PBS supplemented with 5 mM calcium chloride. EMLOCs were incubated on ice in dissociation solution and triturated with a P-1000 pipette every 30-60 s for 8 min. Dissociation to single cells was verified by optical inspection and the reaction was terminated by addition of 1 ml ice-cold 1×PBS with 10% fetal bovine serum (FBS). Cells were pelleted by centrifugation at 1,200×g for 5 min, resuspended in fresh 1× PBS/10% FBS, counted, and centrifuged once more. Supernatant was aspirated completely and cells were resuspended in CryoStor CS10 cryopreservation medium to a final concentration of 1×10⁶ cells per ml, filtered through a 40 μm cell strainer, and transferred to a 1.8 ml Nunc cryo-storage tube. Cells were frozen at −80° C. overnight and transferred to a liquid nitrogen dewar. When samples from both time points were dissociated and stored, samples were shipped overnight on dry ice to University of Buffalo Genomics and Bioinformatics Core at the New York State Center of Excellence in Bioinformatics and Life Sciences.

Single-cell sequencing with CellPlex, cluster annotation and analysis: When samples were received, they were immediately stored at −80° C. On the day of cell capture for sequencing, day 7 and day 16 EMLOC samples were thawed in a 37° C. water bath. Individual time point samples were transferred to separate 15 ml tubes. RPMI1640+10% FBS pre-warmed media was added dropwise to a final volume of 10 ml per tube. Cells were centrifuged at 300×g for 5 min. This washing procedure was performed a total of three times. After the final wash, medium was completely removed and cell samples were separately resuspended in 100 μl of Cell Multiplexing Oligo (10× Genomics). The two populations were suspended with two different oligos as directed by the manufacturer's instructions. After a brief incubation, cells were washed 3× with ice cold 1×PBS (pH 7.4)+1% bovine serum albumin (BSA). Cells were then resuspended in 250 ul 1×PBS/1% BSA and counted on a Logos Biosystems LUNA II in bright field mode with 0.4% trypan blue. The two cell populations with different barcodes were then pooled to 10,000 cells (5,000 from each time point) and recounted. The combined single cell suspension was provided as input for the 10× Genomics Single Cell v3.1 protocol with Feature Barcode technology. After libraries were prepared, they were loaded onto an Illumina NextSeq in high-output mode with a general target of 50,000 reads per cell to provide for sufficient depth and transcriptomic saturation. Post sequencing, data was demultiplexed and provided as input into the 10× Genomics Cell Ranger multipipeline (ver 4), which quantifies the transcriptomic profile of each cell against a reference genome. Sequence saturation, detected barcodes per cell, percent of transcripts in cell, and general alignment statistics were evaluated for quality. Cell Ranger matrix files were then used as input into the R Bioconductor package Seurat (ver 4). Cells with outlier-status, abnormal gene detection rates, and high mitochondrial transcript load that is an indicator of cellular stress were filtered from the analysis. After filtering, the data was underwent Seurat normalization and integrated using the SCTransform and integration protocol, followed by UMAP (Uniform Manifold Approximation and Projection) or the alternative PHATE dimensionality reduction for visualization (Moon et al., 2019). Using the called clusters, cluster-to-cluster differential expression testing using the Wilcoxon Rank Sum Test was used to identify cluster-defining biomarkers, as well as further exploratory analysis with known biomarker genes (gene lists provided in Table S1). Data was then exported from Seurat for further analysis in the Loupe browser.

TABLE S1
Summary of non-limiting scRNAseq
biomarkers reported in Results:

| Cell type, tissue type or process | Biomarker reported |
|---|---|
| Cell proliferation | MKI67 |
| First heart field | TBX5 |
| First heart field | NKX2-5 |
| First heart field | HAND1 |
| Second heart field | HAND2 |
| Second heart field | MEF2C |
| Second heart field | TBX18 |
| Second heart field | ISL1 |
| Cardiac sarcomere | TNNT2 |
| Cardiac sarcomere | TNNT1 |
| Cardiac sarcomere | MYL7 |
| Fetal heart development | KRT8 |
| Fetal heart development | KRT18 |
| Fetal heart development | APOE |
| Fetal heart development | PLAC9 |
| Fetal heart development | S100A10 |
| Splanchnic mesoderm | FOXF1 |
| Splanchnic mesoderm | PDGFRA |
| Splanchnic mesoderm | TWIST1 |
| Splanchnic mesoderm | PRRX2 |
| Ventricular specification | GATA4 |
| Ventricular specification | GATA6 |
| Ventricular specification | CDH2 |
| Ventricular specification | NFIA |
| Ventricular specification | ISL1 |
| Ventricular specification | FAT4 |
| Ventricular specification | ALDH1A2 |
| Ventricular specification | TTN |
| Left-right asymmetry | IRX3 |
| Left-right asymmetry | HAND1 |
| Left-right asymmetry | PITX2 |
| Left-right asymmetry | RTTN |
| Cardiac neural crest cells | ETS1 |
| Cardiac neural crest cells | EDNRA |
| Cardiac neural crest cells | TGIF1 |
| Cardiac neural crest cells | HOXA3 |
| Rapid ventricular conduction | IRX3 |
| Cardiac repolarization | IRX5 |
| Calcium flux | ITPR2 |
| Calcium handling | SLC8A1 |
| AV conduction, organization | GJA1 |
| AV conduction, organization | CACNA1H |
| AV conduction, organization | TBX3 |
| AV conduction, organization | CXCL12 |
| AV conduction, organization | DSP |
| Atrioventricular valves | POSTN |
| Atrioventricular valves | NPR3 |
| Atrioventricular valves | TBX3 |
| Atrioventricular valves | NFATC4 |
| Outflow tract smooth muscle | CNN1 |
| Outflow tract smooth muscle | TAGLN |
| Outflow tract | ISL1 |
| Outflow tract | PDE5A |
| Outflow tract | CDH11 |
| Outflow tract | FN1 |
| Outflow tract | MEGF6 |
| Outflow tract | MSX2 |
| Outflow tract | SEMA3C |
| Outflow tract | EMILIN1 |
| Cardiac jelly | VCAN |
| Cardiac jelly | ADAMTS1 |
| Cardiac jelly | ANGPT1 |
| Vascular endothelial cells | KDR |
| Vascular endothelial cells | FLT1 |
| Vascular endothelial cells | ESAM |
| Vascular endothelial cells | CDH5 |
| Vascular endothelial cells | GJA4 |
| Epicardial cells | WT1 |
| Epicardial cells | TCF21 |
| Epicardial cells | TPJ1 |
| Epicardial cells | LHX2 |
| Epicardial cells | LHX9 |
| Epicardial cells | TBX18 |
| Epicardial cells | PLAC9 |
| Cardiac fibroblast | IFI16 |
| Cardiac fibroblast | TBX5 |
| Cardiac fibroblast | IGFBP5 |
| Cardiac fibroblast | BTS2 |
| Extracellular matrix | COL1A1 |
| Extracellular matrix | COL1A2 |
| Extracellular matrix | COL3A1 |
| Extracellular matrix | COL6A3 |
| Extracellular matrix | FN1 |
| Extracellular matrix | DCN |
| Extracellular matrix | EMILIN1 |
| Extracellular matrix | PLAC9 |
| Extracellular matrix | LAMA1 |
| Extracellular matrix | LAMA4 |
| Extracellular matrix | LAMA5 |
| Foregut endoderm | FOXA2 |
| Foregut endoderm | NKX2-1 |
| Foregut endoderm | EPCAM |
| Foregut endoderm | CDH1 |
| Foregut endoderm | HHEX |
| Foregut endoderm | SHH |
| Trunk neuroectoderm | SOX2 |
| Trunk neuroectoderm | FABP7 |
| Trunk neuroectoderm | ZIC1 |
| Trunk neuroectoderm | RFX4 |
| Trunk neuroectoderm | HES5 |
| Trunk neuroectoderm | EDNRB |
| Trunk neuroectoderm | NTRK2 |
| Trunk neuroectoderm | OLIG3 |
| Trunk neuroectoderm | MSX1 |
| Trunk neuroectoderm | HOXC6 |
| Trunk neuroectoderm | HOXC9 |
| Neuronal | STMN2 |
| Neuronal | GAP43 |
| Neuronal | TUBB3 |
| Neuronal | ELAVL3 |
| Neuronal | DLG4 |
| Neuronal | CAMK2A |
| Neuronal | SLC18A3 |
| Neuronal | SLC17A6 |
| Neuronal | CHRNA3 |
| Neuronal | NTRK3 |
| Sensory neuron | POU4F1 |
| Motor neuron | MNX1 |
| Autonomic neuron | PHOX2B |
| Autonomic neuron | ASCL1 |
| Sympathetic neuron | INSM1 |
| Sympathetic neuron | ISL1 |
| Schwann cell glia | SOX10 |
| Schwann cell glia | PLP1 |
| Schwann cell glia | MPZ |
| Schwann cell glia | S100B |
| Schwann cell glia | TFAP2B |
| Schwann cell glia | NGFR |
| Neuro-cardiac patterning | NPY |
| Neuro-cardiac patterning | BDNF |
| Neuro-cardiac patterning | SEMA3A |
| Neuro-cardiac patterning | PRPH |
| Neuro-cardiac patterning | EDNRA |
| Neuro-cardiac patterning | ISL1 |
| Renal | MAL |
| Renal | SIM1 |
| Renal | EPCAM |
| Renal | TFAP2A |
| Renal | SIX1 |
| Renal | WT1 |
| Renal | ITGA8 |
| Renal | EYA1 |
| Renal | PAX2 |
| Renal | PAX8 |
| Renal | LHX1 |
| Renal | EMX2 |
| BMP signaling | BMP4 |
| BMP signaling | BMP7 |
| WNT signaling | WNT2B |
| WNT signaling | WNT1 |
| WNT signaling | WNT3A |
| Hox code | HOXA4 |
| Hox code | HOXC9 |
| Hox code | HOXD8 |
| Cadherin | CDH1 |
| Cadherin | CDH6 |
| Cadherin | CDH11 |

Calcium Imaging Of Contractility: The EMLOCs were incubated with Fluo-4 AM dye as described above in 1 ml of medium for 30 min. Cells were rinsed once in HBSS and imaged in BrainPhys medium without Phenol red. Timelapse series were acquired at 50 ms exposure using a 488 nm LED at 200 ms intervals for 1.5 min duration. Analysis of calcium spike transients was performed using ImageJ. A wide field fluorescence microscopy was performed using a Zeiss Axio Observer.Z1 inverted fluorescence microscope (20×/0.8 air objective for live cell calcium imaging). Images were acquired using an Hamamatsu ORCA ER CCD camera and Zeiss AxiovisionRel software (ver. 4.8.2).

Phase Contrast And Whole Mount Immunofluorescence: Phase contrast microscopy was performed at room temperature directly in the biosafety hood. Images were acquired using a Zeiss Invertoskop 40C (5×/0.12 CP-Apochromat, 10×/0.25 Ph1 A-Plan and 20×/0.30 Ph1 LD A-Plan, 40×/0.50 Ph2 LD A-Plan) mounted with an Olympus DP22 color camera and cellSens acquisition software. Whole-mount immunofluorescence preparation was performed as previously described (Veenvliet et al., 2020; Olmsted and Paluh, 2021). EMLOCs were pooled on the day of fixation, rinsed once with 1× phosphate-buffered saline (PBS), and fixed in 10% neutral buffered formalin solution at 4° C. for 2 h. Samples were washed three times in 1×PBS for 5 min at room temperature. Samples were then permeabilized by three successive incubations in 0.2% Triton X-100 in 1×PBS (PBST) for 20 min at 4° C., and blocked overnight in 1% BSA in PBST. For primary antibody incubation, samples were distributed evenly to 12-well plates in 1 ml blocking solution per well. Primary antibodies were added to requisite dilutions in 1% BSA (1×PBS): anti-SOX2 (goat, 5 μg/ml); anti-GATA4 (5 μg/ml); anti-GATA6 (5 μg/ml); anti-CDH1/E-Cadherin (5 μg/ml); anti-FOXA2 (5 μg/ml); anti-β-III-tubulin (rabbit, 1:2,000); phospho-Tau Ser214 (rabbit, 1 μg/ml); CDH2/N-Cadherin (1:200); anti-Collagen Type 1 (1:500, 1 mg/ml stock); anti-Laminin (1:500, 1 mg/ml); anti-Desmin (5 μg/ml); anti-Cardiac Troponin-T (25 μg/ml). Plates were left rocking at 4° C. for 24-48 h, rinsed three times in blocking solution, then three times in PBST for 5 min each at room temperature (2 ml centrifuge tubes). Secondary antibodies were incubated 1:1,000 with 2 drops of NucBlue fixed cell stain (Invitrogen) directly in the 2 ml tubes overnight at 4° C. Goat anti-mouse Cy5 secondary antibody was added the next day following washes steps to dilute donkey anti-goat AlexaFluor 594 secondary antibody for samples stained with three antibodies (mouse, rabbit, goat). Samples were again incubated overnight rocking at 4° C. Stained and rinsed EMLO samples were post-fixed in 10% neutral buffered formalin for 20 min at 4° C., and equilibrated in 0.1 M phosphate buffer (PB: 0.025 M NaH₂PO₄, 0.075 M Na₂HPO₄, pH 7.4) containing 0.2% Triton-X 100 by three successive incubations of 5 min at room temperature. To clear samples, 0.1 M PB was aspirated and replaced with 100 μl of 88% Histodenz solution (w/v) dissolved in 0.2 M PB and filter sterilized. Samples were left in the dark at 4° C. for 24 h, mounted on glass slides and sealed in clear nail polish for imaging. Samples were imaged on a Leica confocal TCS SP5 II system in conjunction with Leica Application Suite Advanced Fluorescence software. The SP5 II system was equipped with 10×/0.30 HCX PL FLUOTAR air, 20×/0.70 HC PL APO CS air or immersion, and 40×/1.25 HCX PL APO immersion objective lenses. Complete or partial Z-stacks were acquired at ˜2-2.5 μm separation distance. If necessary, images were corrected linearly for brightness in ImageJ. Maximally projected Z-stacks were performed directly in the Leica software and exported, or were made using Z-project in ImageJ.

Quantification Of Immunofluorescence Signal: Quantification immunofluorescence signals along the anterior-posterior axis was performed as described (Rossi et al., 2021). cTnT signal was quantified from maximal projection images (z-axis) and the FOXA2 was quantified from single Z-slices in order to capture the gut tube. The anterior-posterior axis length was measured from pole-to-pole for each gastruloid. Fluorescence intensity was determined using the plot profile tool in Fiji ImageJ, and was normalized along with gastruloid length to enable comparative analysis. FOXA2 and cTnT mean curves were plotted in GraphPad Prism 9 and juxtaposed. Curves were smoothed using a LOWESS function in GraphPad. Only the single channels in question were quantified.

Statistical analysis and reproducibility: Microsoft Excel (v16.16.27) and GraphPad Prism 9 (v9.0.2) were used for statistical analysis and data plotting. Data are reported as (mean+/−s.e.m.), analyzed using paired or unpaired two-tailed t-test as indicated. ****p<0.0001, ***p<0.001, **p<0.01, *p<0.05, n.s. not significant (α=0.05 threshold for significance). Power analysis was not performed. Detailed information for each experiment is provided in Results and Figure Legends. Key resources including primary antibodies, chemicals and other reagents, software and equipment, and commercial kits are provided.

Angiocrine And Pro-Cardiogenic Factors Redirect Multi-Lineage EMLO Gastruloids For Human Developmental Cardiogenesis (EMLOCs): EMLO gastruloids were previously generated with co-developing central and peripheral neurons and trunk mesendoderm including components of the enteric nervous system (Olmsted and Paluh, 2021a, 2021b). To test the ability of EMLOs to model human developmental cardiogenesis (EMLOCs), exposure to growth factors was modified during early formation and polarization stages in a revised protocol (FIG. 1A, FIG. S1). EMLOCs were generated with the hiPSC line H3.1.1 that was previously validated for differentiation into contractile cardiomyocytes (Tomov et al., 2016) and that forms EMLOs by the original protocol described (Olmsted and Paluh, 2021a, 2021b). EMLOCs were handled identically to EMLOs during a 2D induction phase up to 48 h post-aggregation in shaking cultures. At 48 h, the N2B27 supplemented with 10 ng/ml FGF2, 2 ng/ml HGF, and 2 ng/ml IGF-1 in EMLOs was instead supplemented with 30 ng/ml FGF2, 5 ng/ml VEGF, and 0.5 mM ascorbic acid (AA) (FIG. S1A-C) as was done for the mESC cardiac gastruloid model (Rossi et al., 2021). The new cardiogenic factors were added in the absence of the initial factors used for induction. The EMLOC gastruloids were maintained in this pro-cardiogenic medium to day 7. In the first 48 h prior to the medium change, early germ layer biomarkers were expressed in appropriately sized gastruloids as expected for the EMLO protocol. That is, uniform expression of SOX2 24 h post-aggregation, with little to no expression of GATA6 and heterogenous expression of FOXA2 (FIG. S1D). Mitotically active cells in the gastruloid were visible (FIG. S1E). After the medium change, the EMLOC gastruloids that were continually maintained in shaking culture began to elongate by day 5. By day 7, elongated gastruloids formed thin-walled, dilated chamber-like structures with spontaneous contractility (See e.g., FIG. 1). Partitioning of cardiogenic chamber precursors was visible by phase contrast microscopy (FIG. S1F), and validated by expression of the early cardiogenic transcription factor GATA6 along with cardiac Troponin T (cTnT) (FIG. S1G). Multiple distinct cavities resembling chambers were also identified by immunofluorescence in single EMLOCs with early evidence of septation (FIGS. 1B and 1C). These findings support the ability to direct cardiogenic tissue precursors within gastruloids by early manipulation of growth conditions and signaling factors, demonstrated by the genesis of self-organizing cardiogenic compartments. A single cell RNA sequencing (scRNAseq) was subsequently performed to further delineate the cell and tissue precursor types generated by this protocol.

EMLOC Gastruloids Generate Diverse Embryonic Cell Types Of The Human Trunk Revealed By scRNAseq Analysis: Single cell sequencing of H3.1.1 derived EMLOCs was performed at two time points that are day 7 and day 16 after initial aggregation in shaking culture. The integrated dataset of both time points was analyzed (2,859 cells) (FIGS. 1D-1F) along with each time point individually (day 7: 1,004 cells; day 16: 1,855 cells) (FIG. 2, FIG. S2). The integrated dataset was generated in Seurat and visualized using UMAP and PHATE methods. Ten clusters were produced and were annotated using differentially expressed genes (DEGs) and cell or tissue type characteristic biomarkers from the literature (FIGS. 1D and 1E). The top five DEGs for each cluster of the integrated dataset is provided (FIG. 1F) in addition to a comprehensive list of DEGs and genes used for cluster annotation. Clusters were annotated as trunk neuroectoderm/spinal cord progenitors (cluster 1), trunk neurons (cluster 8), peripheral glia/Schwann cells (cluster 10), mitotic cells (cluster 5), mixed cell types (cluster 0), cardiomyocytes (CM; cluster 3), epicardial cells and cardiac fibroblasts (EP, CF; clusters 2 and 7), intermediate mesoderm and metanephric mesenchyme (IM; cluster 4), genitourinary/renal epithelium (GU; cluster 6), and vascular endothelium (VE; cluster 9). By PHATE analysis of day 16 cells, distinct cardiac, epithelial and neural lineages were visualized (FIG. 2A). Important signaling pathways were overlaid including BMP, SHH, and WNT signaling that were delineated along distinct lineages (FIG. 2B). BMP4 was highly expressed in the cardiac region while BMP7 bifurcated along neural and trunk epithelial lineages. SHH was upregulated in a region within cluster 0 of anterior foregut (AFG) phenotype (FOXA2, NKX2-1, SHH, EPCAM) that is a known developmental organizer of cardiogenesis (Anderson et al., 2016). WNT2B expression localized to the cardiac region while WNT1 and WNT3A localized to the spinal cord region with known involvement in neural tube morphogenesis and neural crest patterning. WNT1 is shown. Cadherin and HOX genes were also delineated along respective lineages, consistent with developmental cadherin and HOX codes in vivo (FIG. 2C). CDH11 was upregulated in the cardiac region while CDH6 was upregulated in neuroectoderm and mesenchyme and CDH1 was upregulated in epithelium. Distinct expression of HOXC6 and HOXC9 in neural clusters was indicative of caudal neuraxis and trunk spinal cord. HOXD8 and HOXD9 were specific to epithelium with renal mRNA expression phenotype (cluster 6) while HOXA4 was predominantly expressed in the cardiac region. This scRNAseq analysis reflects a diversity of cell and tissue precursor types generated within EMLOCs with signaling networks, adhesion proteins, and transcription factors mirroring in vivo development. Specific features of the annotated clusters are elaborated on throughout the manuscript where indicated, with emphasis on neural, cardiac, and foregut endodermal lineages.

Multiple Derivatives Of Splanchnic Mesoderm In EMLOCs Identified By scRNAseq: Cardiogenic mesoderm gives rise not only to working cardiomyocytes but also contributes to epicardium, endocardium, connective tissue, outflow tract, valves and the conduction apparatus. Cluster annotation and analysis of scRNAseq data in day 7 and day 16 EMLOCs identified diverse cell types involved in cardiogenesis arising from splanchnic mesoderm (FIGS. 2D-2E, FIG. S3) including cardiomyocytes that form myocardium, cells of the proepicardium and epicardium, and cardiac fibroblasts, in addition to endocardium and vascular endothelium. In mouse, cardiac precursors arise within the splanchnic mesoderm and differentiate into cardiomyocytes by assembling contractile machinery (Tyser et al., 2016). EMLO gastruloids were previously shown to contain splanchnic mesoderm permissive to cardiomyocyte differentiation (Olmsted and Paluh, 2021a). Similarly, EMLOCs retain characteristic expression of the splanchnic mesoderm biomarkers FOXF1, PDGFRA, TWIST1, and PRRX2 (FIG. S3A). GATA4 and GATA6 that are indispensable for cardiogenesis were expressed in a similar distribution (FIG. S3B). Sarcomeric proteins associated with cardiomyocytes such as TNNT2 (cardiac troponin), TNNT1 (slow type troponin) and MYL7 were most localized to cluster 3 at day 16 along with first heart field (FHF) and second heart field (SHF) biomarkers TBX5 and HAND2 (FIG. 2D). Additional FHF genes (NKX2-5, HAND1) and SHF genes (MEF2C, ISL1, TBX18) were similarly distributed. At the day 16 time point, cardiomyocytes were primarily of ventricular cell phenotype. DEGs involved in regulating ventricle growth, morphogenesis, and contractility were identified (FIG. S3B). Cluster 3 contained additional upregulated genes involved in fetal heart development such as dimeric KRT8/KRT18, APOE, PLAC9 and S100A10. These genes were also upregulated in cluster 2 that were annotated as epicardial (EP) cells based on expression profile (WT1, TCF21, TPJ1, LHX2, LHX9, TBX18, PLAC9) (FIG. 2E). Cluster 7 had a similar expression profile to cluster 2, but with several distinguishing features such as reduced proliferation assessed by MK167, upregulation of IGFBP5 associated with cardiac fibroblast activation and BTS2 that interacts with IFI16, a biomarker of mature cardiac fibroblasts. A previous analysis of human fetal cardiogenesis revealed subclusters of actively proliferating, early fibroblasts and those enriched for ECM organization with less proliferation (Cui et al., 2019). A similar observation was made here between cluster 2 (high proliferation by MKI67) and cluster 7 (low proliferation by MK167). The scRNAseq clusters representing cardiogenesis expressed a characteristic milieu of cardiomyocyte and fibroblast-derived ECM genes (FIG. S4). Cluster 0 contained upregulated genes indicating a mixture of cell types from cardiogenic mesoderm including contractile cells (TNNT2, MYL7), outflow tract cells (PDE5A, ISL1, FN1, MEGF6, MSX2, SEMA3C, EMILIN1, CNN1, TAGLN), and cells involved in atrioventricular conduction and organization (GJA1, CACNA1H, TBX3, CXCL12, DSP). Anterior foregut progenitor cells were also identified (HHEX, SHH, FOXA2). Cells expressing cardiac neural crest biomarkers (ETS1, EDNRA, TGIF1, HOXA3) were dispersed throughout the four clusters (FIG. S3C). Cardiac neural crest cells in vivo play critical roles in cardiogenesis and organization, including valve and outflow tract contributions. Regulatory subnetworks within EMLOCs therefore generate a range of the cell types involved in cardiogenesis along with an appropriate ECM milieu.

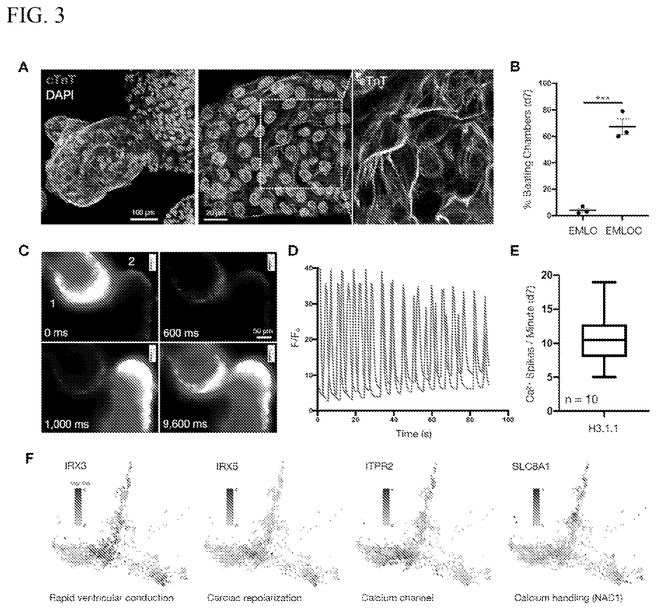

Human EMLOC Multi-Lineage Gastruloids Form Chamber-Like Structures With Spontaneous Contractility And Calcium Signaling: Intracellular changes in Ca²⁺ couple cardiomyocyte depolarization with contraction. To demonstrate that EMLOCs express calcium-regulated contractile proteins and achieve calcium-mediated contractility, we performed 3D fixed and live cell imaging analysis (FIG. 3). The cardiogenic compartment in EMLOCs exhibited visible sarcomeres using cTnT immunofluorescence (FIG. 3A) and spontaneous contractility (FIG. 3B). To quantify spontaneous contractility, we compared beating phenotypes in EMLOC-directed gastruloids versus the original EMLO protocol that is not optimized for cardiogenesis (Olmsted and Paluh, 2020a) (N=3 repeat experiments, ***p=0.0005, t=10.40 df=4 by unpaired two-tailed t-test). We further performed live calcium imaging with Fluo-4 AM (FIGS. 3C-3E). In FIG. 3C, we demonstrate robust Fluo-4 AM activity in the contractile region of two gastruloids and quantify F/Fₒ calcium transient spikes (FIG. 3D). The EMLOCs shown were captured in the same field and are representative of the quantified population. Median calcium transients per min (corresponding to beat frequency) was 10.5 (n=10, max=19, min=5, median=10.5, q1=8, q3=12.75) (FIG. 3E). Notably, this is ˜8× lower than the calcium transient frequency described in cardiogenic gastruloids formed from mESCs (Rossi et al., 2021) and mimics the average human resting heart rate (60 beats per min) that is ˜8× lower than formouse (500-700 beats per min). Known genes involved in cardiac action potential conduction and calcium handling are expressed, including IRX3 and IRX5 that play roles in rapid ventricular conduction and cardiac repolarization, respectively, along with the ITPR2 calcium channel and sodium-calcium exchanger SLC8A1/NAC1 that were all expressed with similar distribution (FIG. 3F). Together these data demonstrate that morphological cardiogenesis chamber features, calcium signaling and spontaneous contractility can be directed in human EMLOCs by modifying the EMLO protocol with exposure to angiocrine and pro-cardiogenic growth factors. As well, we note that similar growth factor regulatory pathways drive cardiogenesis, while reflecting species specific functional outcomes.

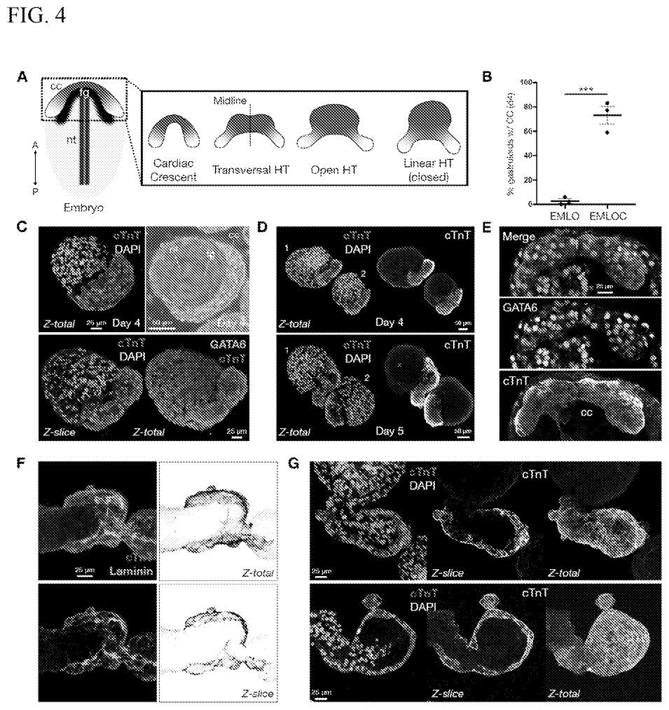

Recapitulating Early Morphogenesis Events In Human Developmental Cardiac EMLOC Gastruloids: The first cardiogenic structure to form in the anterior aspect of the mammalian embryo is called the cardiac crescent, which fuses to form the transversal heart tube that seals dorsally to generate a closed tube with outflow tracts (FIG. 4A). Cardiac crescent-like structures were identified at high penetrance in EMLOC (73.0+/−7.2%, mean+/−s.e.m.) versus EMLO generated gastruloids (2.7+/−1.8%; N=3 repeat experiments; ***p=0.0007, t=9.474, df=4 by unpaired two-tailed t-test) (FIG. 4B). Cardiac crescent regions at day 4 in EMLOC formation contained cTnT+ cardiomyocyte progenitors co-localized with GATA6, a transcription factor required for high fidelity cardiogenesis (FIGS. 4C and 4E). The cardiogenic region increased in size with time, extending laterally away from the main body of the gastruloid (FIG. 4D). The same region developed a cell-free interior, resembling early heart tube formation (FIGS. 4A and 4F) along with cardiac chamber morphological precursors (FIG. 4G).

EMLOC Gastruloid Cardiac Morphogenesis Occurs Anterior To Primitive Gut Tube Endoderm: The developing anterior foregut derived from endoderm has been shown to be essential for cardiogenesis in multiple organisms through crosstalk with splanchnic mesoderm and by providing mechanical cues (FIG. 5A) (Nascone and Mercola, 1995; Varner and Taber, 2012; Anderson et al., 2016; Kidokoro et al., 2018; Han et al., 2020). It has been demonstrated that the primitive gut tube can self-organize reproducibly in the EMLO gastruloid system (Olmsted and Paluh, 2020a). Therefore, whether this structure is present in the EMLOC gastruloids suitable for cardiogenesis was investigated. In mouse, this occurs anterior to the gut tube, including cuboidal epithelialization that is required for second heart field contributions to heart tube formation (Ivanovitch et al., 2017; Cortes et al., 2018). The splanchnic mesoderm is an established and reproducible feature of EMLO and EMLOC formation and also contributes to the gut tube (FIGS. S3A and S3B) (Olmsted and Paluh, 2021a). Using scRNAseq, a population of cells with anterior foregut identity (FOXA2, NKX2-1, SHH, EPCAM) was identified and clustered adjacent to the developing cardiac region (FIG. 2A). Spatially organized FOXA2+ cells adjacent to the cardiac region were also identified using immunofluorescence. By normalizing fluorescence and gastruloid end-to-end length, an average profile for cTnT (cardiac) and FOXA2 (gut tube endoderm) was generated over the anterior-posterior axis to depict relative positioning in day 4 EMLOCs (FIG. 5B) (N=5 EMLOCs). Representative cTnT/FOXA2 immunofluorescence Z-slices are provided for day 5 and day 6 EMLOCs, in addition to CDH1 (E-Cadherin) and GATA6/Type 1 Collagen (FIGS. 5C and 5D). These data prioritize visualization of gut tube positioning relative to the cardiogenic region, demonstrating the appropriate posterior embryological trunk spatial organization with respect to cardiogenesis. We performed immunofluorescence imaging with cTnT and the proliferation marker Ki67, as well as CDH2 that is essential for ventricular wall morphogenesis (Miao et al., 2019). The co-immunofluorescence of the CDH2 biomarker with cTnT further revealed early organization of cells in the cardiac crescent into epithelial-like cytoarchitectures that is a contributing factor in heart tube formation (FIG. 5E) (Cortes et al., 2018). This pattern was also observed by scRNAseq co-expression patterns of CDH2 with ventricular biomarkers (FIG. S3B).

Whether EMLOCs recapitulate distinct phases of cardiomyocyte differentiation and morphogenesis that are ongoing developmentally (FIG. S5) was investigated. As done in vivo (Ivanovitch et al., 2017), cardiomyocyte shape was used to indirectly infer distinct phases of cardiomyocyte differentiation versus structural morphogenesis with cellular proliferation. Cardiomyocyte cell shape was characterized as rounded (morphogenesis and proliferation; FIG. S5A top) versus adhesive flat/mosaic (differentiation; FIG. S5A bottom) and quantified the relative proportion of EMLOCs exhibiting one phenotype or the other in single day 7 fixed samples (FIG. S5B) (N=4 replicates; 34+/−18% round, range 16 to 59%; 66+/−18% flat/mosaic, mean+/−s.e.m., range 41 to 84%; n.s. p=0.1776, t=1.754, df=3 by paired two-tailed t-test). Together, these data demonstrate that features of in vivo cardiogenesis can be modeled in EMLOCs within the appropriate multi-lineage gastruloid microenvironment.

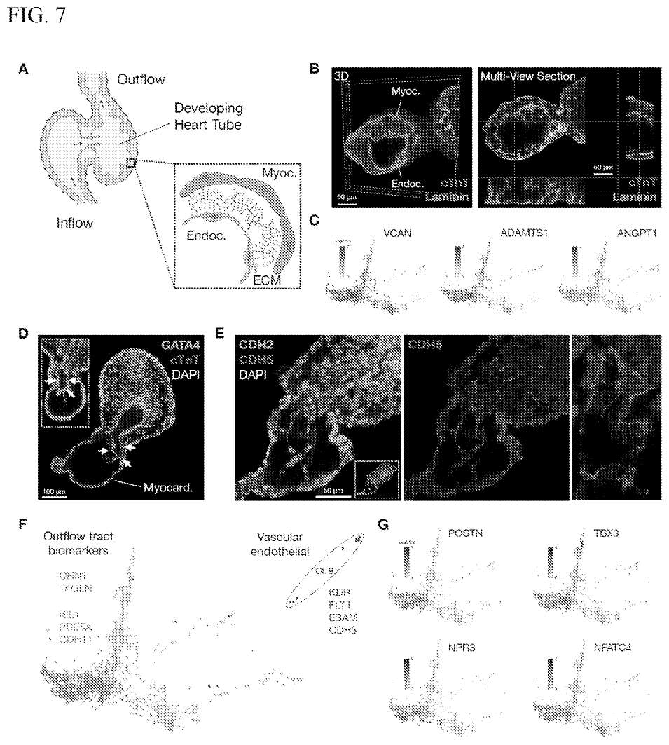

EMLOCs exhibit specialization over heart tube length and multi-layering of chamber walls during morphogenesis: As the cardiac crescent is remodeled into the contractile primitive heart tube in vivo, specialization over the length of the tube establishes the future blueprints for the adult heart in terms of septated chambers and outflow tracts that transmit and receive blood (FIG. 6A). Divisions of the embryonic heart are separated by minor constrictions in the tube. Day 7 EMLOCs with cardiogenic compartments were identified resembling this stage in heart tube development (FIG. 6B). After day 7, constricted tubes became dilated and had early divisions between chamber precursors (FIGS. 6C and 6D), visualized by 3D reconstructions and multi-dimensional analysis. The fluid-filled contractile cavities were completely surrounded by continuous cTnT+ cardiomyocytes indicative of myocardium. In addition, the cavities had open channels communicating with the posterior EMLOC compartment (FIGS. 6C and 7D, FIG. 7). Genes involved in left-right asymmetry specification during in vivo cardiogenesis were also upregulated in the day 16 scRNAseq data set (IRX3, HAND1, PITX2, RTTN).

In developing heart chambers in situ, the chamber walls are multi-layered, with myocardium composed of working contractile and conducting cardiomyocytes comprising the outermost layer, and endocardium lining comprising the innermost layer (FIG. 7A). An epicardial membrane surrounds these layers. Myocardium and endocardium are initially separated by ECM-rich “cardiac jelly” (Kim et al., 2018) that is degraded with time, necessary for chamber morphogenesis. Using 3D image reconstruction and multi-dimensional analysis, we show that chamber wall organization was recapitulated in developing EMLOC chamber-like structures (FIG. 7B). We identified gene biomarkers of cardiac jelly ECM and its spatiotemporal degradation VCAN, ADAMTS1, and ANGPT1 that were highly expressed in day 16 EMLOCs (FIG. 7C) (Kim et al., 2018). Notably, an interior channel partially lined by cTnT+ cells was also identified with high penetrance, postulated here to be initiation of the putative outflow tract due to its appropriate positioning (FIG. 7D) and that is lined by segmental CDH5 immunostaining extending out into the posterior compartment of the EMLOC (FIG. 7E). By scRNAseq, we identified cells with combinations of biomarkers for smooth muscle (CNN1/TAGLN), outflow tract development (ISL1/PDE5A/CDH11), and well-differentiated vascular endothelium (cluster 9; KDR/FLT1/ESAM/CDH5) (FIG. 7F). Nodal and valvar biomarkers were also present with a similar distribution (POSTN/TBX3/NPR3/NFATC4) (FIG. 7G). These data, taken together, are consistent with cardiogenesis in EMLOCs proceeding in an appropriate spatiotemporal manner and detailed morphological and gene expression changes, according to aspects of in situ development.

EMLOCs capture neurogenesis within a neuro-cardiac model of human trunk development: The EMLO approach (Olmsted and Paluh, 2021a, 2021b) was developed to study early neurogenesis events in trunk development. To investigate early neural lineage biomarkers in EMLOCs immunofluorescence and scRNAseq was performed (FIG. 8). TUJ1 immunostain was first identified in cells opposite the anterior cardiac domain with low level staining, typical of neural stem/progenitor cells that express this protein at lower levels (FIG. 8A). The initial emergence of neurons at day 7, identified by morphology and biomarkers, parallels that seen for the original EMLO protocol (Olmsted and Paluh, 2021a, 2021b). In EMLOCs, the posterior region of neurogenesis emerged from one to several SOX2+/TUJ1+ neuroectodermal rosettes and the number of neurons increased significantly overtime (FIGS. 8B-8D). Given the relatively low number of neurons present at the day 7 time point when spontaneous contractility is already occurring, it is unlikely that neuronal function plays a significant signaling role in initiating spontaneous cardiogenic contractions at this early stage. The increase in the number of TUJ1+ neurons in EMLOCs was quantified between days 7 and 18 (FIG. 8E) (day 7: 5+/−2 TUJ1+ neurons, mean+/−s.e.m; day 18: 218+/−24 TUJ1+ neurons; ****p<0.0001, t=8.929, df=18 by unpaired two-tailed t-test). The proportion of EMLOCs was quantified with a neuronally integrated cardiac compartment between days 7 and 25 (FIG. 8F), which similarly increased with time (day 7: 3.3+/−1.9% of population; day 25: 55.8+/−2.9; **p=0.0011, t=12.58, df=3 by paired two-tailed t-test; N=4 replicate experiments). A nidus of neurogenesis from neural rosettes occurred within GATA6+ surrounding mesenchymal-like tissue (FIG. 8G). Notably, the gut tube can be distinguished from surrounding rosettes by a laminated acellular border, whereas the rosettes are more continuous with the adjacent GATA6+ cells (FIG. S6). In EMLOCs, the open channels from the cardiac chambered region connect with the proximal compartment containing neural rosettes.

In the integrated scRNAseq dataset, clusters 1, 8 and 10 predominantly represented the neural lineage. Neural progenitors (cluster 1; ZIC1, RFX4, HES5, FABP7, EDNRB, NTRK2, OLIG3, MSX1) became specialized neuronal subtypes (cluster 8; INSM1, ELAVL3, DLG4, CAMK2A, SLC18A3, SLC17A6, CHRNA3, NTRK3), and a population of neural crest-derived Schwann cells was present (cluster 10; SOX10, PLP1, MPZ, S100B, TFAP2B, NGFR) (FIGS. 1D-1F, FIG. 8H). Combined INSM1/ISL1 expression indicates that sympathetic neurogenesis occurs in EMLOCs, which is particularly relevant to developing cardiac innervation. Restricted expression of HOXC6 and HOXC9 to these clusters supports spinal cord and trunk identity. Schwann cells increased ˜16-fold from the day 7 to day 16 time point. As well, biomarkers of specialized neuronal subtypes that were lacking at day 7 began to emerge. The transcriptional phenotype for differentiating autonomic neurons that is ASCL1 (93/151 neurons, ˜62%) and PHOX2B (14/151 neurons, ˜9%) predominated versus sensory neurons POU4F1/BRN3A (38/151 neurons, ˜25%) or motor neurons MNX1/HB9 (6/151 neurons, ˜4%) (FIG. 8H). This finding is distinct from EMLOs (Olmsted and Paluh, 2021a), in which motor neurons were primarily generated.

EMLOCs express biomarkers of cardiac innervation: In parallel with neurogenesis, axonal projections navigating the extracellular space to their target sites are expected to require spatial signals to generate the selective patterning on organs for innervation. Molecular and morphogenic features of the developing heart must therefore play an active role in establishing autonomic innervation, where the proper cellular milieu and receptive fields for innervation will dictate selective neuronal interactions. As such, several genes were identified with known roles in this process that were expressed in the cardiogenic region of the UMAP plot including and that code for neuropeptide Y (NPY), brain-derived neurotrophic factor (BDNF), semaphorin 3A (SEMA3A), peripherin (PRPH), endothelin receptor type A (EDNRA), and ISL-1 (FIG. 8I). Genes involved in autonomic neurogenesis and cardiogenesis such as ISL1 also play a role in development and innervation of cardiac pacemaker cells that dictate automaticity and participate in the conduction system apparatus.

By immunofluorescence, neurons were not identified within the cardiac region at the earlier day 7 and day 8 time points. We therefore analyzed the degree to which cardiogenic and neurogenic regions of the EMLOCs co-develop and integrate. In EMLOCs at day 16 or more in formation, neurons were observed both in the posterior compartment and intercalated with cardiomyocytes anteriorly, resembling in vivo ganglionated plexuses that characterize heart innervation (Ashton et al., 2018) (FIG. S7). In day 25 EMLOCs (FIGS. 8J and 8K), the cardiogenic region remained contractile (FIG. 8J) and neurons formed elaborate structural networks within the myocardium that are excluded from the chambers, appreciated using 3D reconstruction analysis (FIG. 8K). Terminating neuronal fibers on cardiomyocytes were identified in part as axons using the phospho-tau (Ser214) immunostain (Figure S7E). The co-habitation of neurons and cardiomyocytes is anticipated in the same region is a precursor to neuro-cardiac cooperative interactions such as initiation of innervation.

### Example 2

This protocol (See FIG. Ss1 and FIG. Ss4) describes the generation of EMLOCs from the hiPSC line H3.1.1 derived from fibroblasts of a self-designated Hispanic-Latino donor. This low passage hiPSC line was originally published with other ethnically diverse lines (Chang et al., 2015). The protocol is defined by four general stages: (1) 2D induction of hiPSC colonies, (2) transition to shaking culture and EMLOC early polarization, (3) EMLOC cardiac induction, and (4) EMLOC multi-lineage differentiation, chamber morphogenesis and innervation.

Coat Cultureware With hESC-qualified Matrigel Timing: 24 h.: The description is for a 35 mm culture dish: Handle and store hESC-qualified Corning Matrigel according to manufacturer's instructions with attention to preparing and storing aliquots. Thaw 5 mL parafilm-wrapped Matrigel on ice placed within a 4° C. fridge overnight. The next day, 100 μL aliquots of thawed, undiluted Matrigel are prepared on ice and restored at −20° C. for later use. To prepare freshly coated cultureware, remove new 35 mm culture dish and UV sterilize with the top off for 30 min in a laminar flow tissue culture hood. After 30 min UV sterilization, pre-chill dish at −20° C., ˜20 min prior to use. Thaw a single aliquot (100 μL) of Matrigel on ice ˜45 min prior to use. Dilute Matrigel stock 1:100 in ice cold DMEM/F-12 (e.g., 20 μL/2 mL). Add 1 mL of diluted Matrigel per dish. To polymerize surface coating, incubate dish at 37° C. in a humidified incubator with 5% CO₂ for at least 1 h prior to use. The steps should be carried out on ice to prevent premature gelling and/or non-uniform coating of matrix.

Human Induced Pluripotent Stem Cell (hiPSC) Culture: Timing: ˜3-7 days; To thaw hiPSCs cryopreserved in mFreSR cryopreservation medium, transfer 1 vial containing 1 mL of cell suspension from liquid nitrogen storage to 37° C. water bath. While cell suspension is thawing, remove DMEM/F-12 from the freshly coated Matrigel plate, rinse 1× with 1 mL DMEM/F-12, and replace with 2.5 mL mTeSR Plus hiPSC pluripotency medium containing 1× penicillin-streptomycin (mTeSR Plus is supplemented 1× with penicillin-streptomycin unless otherwise specified in this protocol). Before applying mTeSR Plus, bring working volume to room temperature for ˜15 min outside of water bath. In laminar flow tissue culture hood, carefully administer 1 mL of cells in thawed mFreSR dropwise to fresh mTeSR Plus (3.5 mL total), attempting to distribute cells over the surface area of the dish. Incubate seeded cells at 37° C. in a humidified incubator with 5% CO₂ overnight. The next morning, visually inspect for stem cell colony adherence (FIG. Ss2A). Rinse the cells 2× with 1 mL of fresh DMEM/F-12 prewarmed in 37° C. water bath. Replace with 2 mL mTeSR plus prewarmed to room temperature and return to incubator for cell expansion. Follow cells by visual inspection to ensure proper colony density and maintenance of undifferentiated cells and replace fresh mTeSR Plus daily, every other day or every 2 days (double feed volume) following manufacturer's instructions. Any combination of feeding intervals can be used when following these guidelines. For advancing to EMLOC formation, passage hiPSCs 1:6 into Matrigel-coated 6-well plates when hiPSC cultures are stable (˜60% confluency).

Passaging Human Induced Pluripotent Stem Cells: Timing: ˜24 h; Description is for passage from a 35 mm culture dish to 6-well plate. Remove media from the well to be passaged and immediately add 1 mL of Gentle Cell Dissociation Reagent (GCDR) to the empty well. Incubate at room temperature for ˜3 min. The GCDR incubation time requires cell line-specific optimization according to manufacturer's instructions. The incubation time here allows cells to be released in small ‘colony patches’ and not as single cells. Gently aspirate the GCDR without dislodging the cells. Immediately add 3 mL mTeSR Plus to the well that is being passaged. To dislodge colonies, use a 5 mL serological pipette oriented orthogonally to the plane of the plate. Perform a side-to-side scraping motion over the entire area of the well. Rotate the plate 90 degrees and repeat the side-to-side scraping motion to ensure that the bulk of cells are dislodged from the substrate. Mix 2× using a P-1000 blue tip. Gently transfer 0.5 mL of dislodged cells to each well of a Matrigel-treated 6-well plate already containing 1 mL of mTeSR Plus. Cells should be added dropwise quickly and serially to obtain an even distribution in each well (1.5 ml total). Incubate the plate overnight, undisturbed at 37° C. to allow colonies to settle and adhere. The next day, visually inspect cultures for adherence of small colonies. If positive, remove the 1.5 mL mTeSR Plus. Rinse 2× in 1 mL DMEM/F-12 to remove non-adherent cell debris. Add 2 mL fresh mTeSR Plus to each well. Return the plate to the incubator. Add fresh media changes every 2-5 days, until the colonies are expanded, and cultures are ˜50-60% confluent (FIG. Ss2B and FIG. Ss2C). This confluency percentage is optimal for cell induction to generate EMLOCs.

Preparation Of N2B27 Basal Media: Timing: ˜30 min; Prepare 1:1 DMEM/F-12:Neurobasal Plus medium appropriately supplemented to the following final concentrations: 2% (v/v) B-27 Plus, 1% (v/v) N-2, 1× GlutaMAX, 1×MEM Non-Essential Amino Acids, 1× penicillin-streptomycin. For 500 mL N2B27 add 235 mL DMEM/F-12, 235 mL Neurobasal Plus, 10 mL B-27 Plus, 5 mL N-2, 5 mL of 100× GlutaMAX, 5 mL of 100×MEM Non-Essential Amino Acids, 5 mL of 100× penicillin-streptomycin. Use a 0.2 μm pore filter to sterilize this solution and store at 4° C. Prewarm working volumes to room temperature as needed.

### Key Resources Table:

| | | |
|---|---|---|
| REAGENT or RESOURCE | SOURCE | IDENTIFIER |

Antibodies

| | | |
|---|---|---|
| Goat anti-SOX2 | R&D Systems | Cat. No.: |
| | | AF2018; |
| | | RRID: |
| | | AB_355110 |
| Goat anti-GATA6 | R&D Systems | Cat. No.: |
| | | AF1700; |
| | | RRID: |
| | | AB_2108901 |
| Goat anti-FOXA2 | R&D Systems | Cat. No.: |
| | | AF2400; |
| | | RRID: |
| | | AB_2294104 |
| Rabbit anti-β-tubulin | BioLegend | Cat. No.: |
| III/TUJ1 (TUBB3) | | 802001; |
| | | RRID: |
| | | AB_2564645 |
| Mouse anti-cardiac Troponin T | R&D Systems | Cat. No.: |
| | | MAB1874 |
| Goat anti-CDH1/E- cadherin | R&D Systems | Cat. No.: |
| | | AF648; |
| | | RRID: |
| | | AB_355504 |
| Rabbit anti-CDH2/N- cadherin | Cell Signaling | Cat. No.: |
| | Technologies | 13116; |
| | | RRID: |
| | | AB_2687616 |

Chemicals, peptides, and recombinant proteins

| | | |
|---|---|---|
| hESC-qualified Matrigel | Corning | Cat. No.: |
| | | 08-774-552 |
| mTeSR Plus | STEMCELL | Cat. No.: |
| | Technologies | 05825 |
| Gentle Cell Dissociation | STEMCELL | Cat. No.: |
| Reagent | Technologies | 07174 |
| Neurobasal Plus Medium | Thermo Fisher | Cat. No.: |
| | Scientific | A3582901 |
| DMEM/F-12 | Thermo Fisher | Cat. No.: |
| | Scientific | 11320033 |
| N-2 supplement (100X) | Thermo Fisher | Cat. No.: |
| | Scientific | 17502048 |
| B-27 supplement (50X) | Thermo Fisher | Cat. No.: |
| | Scientific | 17504044 |
| GlutaMAX | Thermo Fisher | Cat. No.: |
| | Scientific | 35050061 |
| MEM Non-Essential | Thermo Fisher | Cat. No.: |
| Amino Acids | Scientific | 11140050 |
| Penicillin-Streptomycin | Thermo Fisher | Cat. No.: |
| | Scientific | 15140122 |
| CHIR 99021 | Tocris | Cat. No.: |
| | Bioscience | 4423 |
| bFGF/FGF2 | R&D Systems | Cat. No.: |
| | | 233-FB |
| HGF | R&D Systems | Cat. No.: |
| | | 294-HG |
| IGF-1 | R&D Systems | Cat. No.: |
| | | 291-G1 |
| Y-27632 | Tocris | Cat. No.: |
| | Bioscience | 1254 |
| VEGF 165 | R&D Systems | Cat. No.: |
| | | 293-VE |
| L-ascorbic acid | Tocris | Cat. No.: |
| | Bioscience | 4055 |
| Accutase | STEMCELL | Cat. No.: |
| | Technologies | 07920 |
| Anti-Adherence Rinsing | STEMCELL | Cat. No.: |
| Solution | Technologies | 07010 |
| HBSS CM-free | Thermo Fisher | Cat. No.: |
| | Scientific | 14175079 |

Experimental models: Cell lines

| | | |
|---|---|---|
| H3.1.1 | Paluh lab | Chang et al., |
| | | 2015; Tomov et |
| | | al., 2016 |

Other

| | | |
|---|---|---|
| 6-well plate | CELLTREAT | Cat. No.: |
| | | 229105 |
| Disposable Vacuum Filter/ | Thermo Fisher | Cat. No.: |
| Storage Systems | Scientific | 430773 |
| 100 mm petri dish | Fisher | Cat. No.: |
| | | S33580A |
| CHEMcell VERSA-ORB2 | Chemglass Life | Cat. No.: |
| orbital shaker | Sciences | CLS-4021-100 |

Step-By-Step Method Details: STEP 1: 2D induction of hiPSC colonies for EMLOC formation: The short induction time at this step yields mesendodermal-like cellular starting material that importantly is also primed for neural differentiation. Protocols for trunk biased uniformly committed neuromesodermal progenitors (NMPs) typically rely on more sustained exposure to FGF and CHIR signaling (e.g., 4-5 d induction period) (Olmsted et al., 2020). The induction factors for 2D adherent colonies and for subsequent aggregate formation and polarization were originally identified by the Gouti laboratory to generate neuromuscular trunk organoids using human stem cells (Faustino Martins et al., 2020). These factors were then optimized for elongating multi-lineage organized (EMLO) gastruloids by our laboratory with key protocol changes previously detailed (Olmsted and Paluh, 2021). The original EMLO gastruloids were not optimized for cardiogenesis.

Timing: ˜2 days: hiPSC colonies at ˜50-60% confluency in single wells of a 6-well plate are ready for 2D induction (FIGS. Ss2B and Ss2C); Supplement working volume of N2B27 with 3 μM CHIR 99021 40 ng/mL FGF2 (referred to herein as Induction Medium) and prewarm to room temperature without a water bath. FGF2 regulates differentiation and migration during cell development orchestrating caudalization (Goto et al., 2017), while CHIR 99021 activates the Wnt/pi-catenin signaling via GSK3 inhibition (Naujok et al., 2014). Cell-line dependent induction with CHIR 99021 and FGF2 must be optimized both for concentration and time of exposure. In our hands, the ideal CHIR 99021 concentration window for line H3.1.1 is between 3-3.5 μM for 2-3 d of induction. By visual inspection, optimal colony induction is characterized by slight raised-edge character just as cells begin to migrate away from the colony border (FIG. Ss2D). Also important is consistency of cell numbers being induced so that factor concentrations are appropriate at this step and subsequent steps. Remove the mTeSR Plus medium and rinse cells 2× with DMEM/F-12. Add 2 mL of Induction Medium to the hiPSC wells that will be used to generate EMLOCs. Return plate to incubator. After 24 h, exchange the medium with 2 mL fresh Induction Medium and return to incubator for an additional 24 h.

STEP 2: Transition to shaking culture and EMLOC polarization: Approximately 48 h after induction as adherent 2D colonies, cultures are primed to generate single cell suspensions to form 3D aggregates by orbital shaking. This transition helps to drive formation of uniformly and appropriately sized aggregates and introduces additional mechanical cues during the early polarization stage. Initial starting aggregates with small cell number (˜50-100 □m diameter aggregates; 50-100 cells each) are critical to establish the necessary axis length for local signaling and polarization (van den Brink et al., 2014).

Timing: 2 days: 48 h after the initial induction as 2D colonies in Induction Medium, cells can be used to generate 3D aggregates. Prepare a fresh 6-well plate by treatment with 1 mL Anti-Adherence Rinsing Solution per well for 10 min. Remove the Anti-Adherence Rinsing Solution from the wells and rinse 2× with 1 mL HBSS (CM-free). On the second rinse, do not remove the HBSS to prevent drying. Remove the Induction Medium from the wells containing primed colonies and rinse 2× with 1 mL HBSS (CM-free). For enzymatic dissociation to single cells, dilute Accutase 1:1 with HBSS (CM-free) and add 1 mL per well. Incubate at 37° C. for 5-10 min (cell line-dependent incubation time). After incubation, gently remove dissociation solution and add 1 mL of N2B27 (no supplements) to the empty well. Dislodge the cells using a side-to-side scraping motion over the entire surface area of the well with a serological pipette oriented orthogonally to the surface. Triturate the suspension manually with a P-1000 pipette ˜6× to generate a single cell suspension. Combine 2-3 wells of Accutase-treated hiPSC cells. To enhance collection of all cells, add the entire volume to a 15 mL conical tube and centrifuge at 350×g for 5 min to pellet the single cells. Aspirate the supernatant from the cell pellet and resuspend in EMLOC Polarization Medium (N2B27 basal medium supplemented with 10 ng/mL FGF2, 2 ng/mL IGF1, 2 ng/mL HGF, 50 μM ROCK inhibitor Y-27632) at the appropriate cell density. Remove remaining HBSS from treated wells and transfer cell suspension to the pretreated low adherence well plate. Note: ROCK inhibitor Y-27632 promotes differentiation of hiPSCs into neural crest-like progenitors (Kim et al., 2015). IGF-1 has effects in CNS neural induction by increasing anterior neural transcription factors (Dyer et al., 2016), and HGF stimulates motogenic and morphogenic activities in development via interaction with the C met tyrosine kinase receptor (Desole et al., 2021). The combined suspensions used to generate aggregates for EMLOCs should have ˜2×10⁶ cells total in 2 mL medium (1×10⁶ cells/mL). Too many cells will interfere with correct ratio of factors. Too few cells will prevent any aggregate formation. Total cell number per well may range from 2×10⁶ to 4×10⁶ cells depending on cell line in 2 mL total volume. The high ROCK inhibitor concentration is useful to ensure single cell survival, promote aggregation, and to induce the neural crest cell lineage. Place the plate on an orbital shaker at 80 rpm clockwise in a humidified incubator with 5% CO₂. Visually inspect the orbital shaking cultures at 24 h post-aggregation. At this stage, round aggregates of similar size distribution (˜50-100 μm) should be visible (FIG. Ss2E). To change the culture medium, pool the aggregates in a 15 mL conical tube and allow them to settle by gravity for ˜10 min. Do not let cultures over-settle to avoid aggregate fusion. If there are aggregates present that are much larger than the rest, manually remove the larger aggregates with a P-1000 blue tip. Aspirate one-half volume of the media and replace with fresh Polarization Medium (N2B27 supplemented with 20 ng/mL FGF2, 4 ng/mL IGF1, 4 ng/mL HGF without ROCK inhibitor Y-27632). The concentration of the growth factors is doubled to maintain the same overall concentration in the wells (assuming the original recombinant proteins are still present). Return the culture to the orbital shaker at 80 rpm clockwise in a humidified incubator with 5% CO₂. The rotational speed is key to achieving the initial size of the starting aggregates and may require cell line-specific optimization.

STEP 3: EMLOC cardiac induction: EMLOC cardiogenesis is stimulated with defined angiocrine and cardiogenic factors in combination (FGF2, VEGF 165, ascorbic acid) to recapitulate morphological hallmarks such as thin walled, dilated chamber-like structures with spontaneous contractility. These factors were previously shown to stimulate cardiogenesis in mouse gastruloids (Rossi et al., 2021) and are applied here, adapting our original EMLO protocol (Olmsted and Paluh, 2021) to induce human cardiogenesis within the multi-lineage gastruloid framework. Timing: 5 days, 48 h post-aggregation, initiate cardiac induction by pooling aggregates in a 15 mL conical tube and allow them to settle by gravity for 10 min as described above. Completely aspirate the Polarization Medium and rinse with 10 mL HBSS (CM-free). Let the aggregates re-settle and aspirate the HBSS. Resuspend in Cardiac Induction Medium (N2B27 supplemented with 5 ng/mL VEGF, 30 ng/mL FGF2, 0.5 mM ascorbic acid). Note: VEGF regulates the development of the vascular endothelium and endocardium through the activation of Akt signaling in endothelial cells (Madonna and de Caterina, 2009), and ascorbic acid promotes cardiac differentiation by enhancing the proliferation of cardiac progenitor cells via the MEK-ERK1/2 pathway (Cao et al., 2011). Return cells to the orbital shaker at 80 rpm clockwise in a humidified incubator with 5% CO₂. The Cardiac Induction Medium can be replaced with fresh media at 4-5 d post-aggregation. It is useful to replace procardiogenic media only once to allow intra-aggregate cell-cell and paracrine signaling. Visually monitor cultures for early polarization and cardiac crescent formation (FIG. Ss2F) and formation of contractile chamber-like structures (FIG. Ss2G). Calcium-mediated cardiomyocyte contractility can be verified by live-cell calcium imaging with Fluo-4 AM dye detailed elsewhere (FIG. Ss2H) (Olmsted and Paluh, 2022).

STEP 4: EMLOC Multi-Lineage Differentiation, Chamber Morphogenesis And Innervation: After 7 d post-aggregation, Cardiac Induction Medium is replaced with non-supplemented N2B27. This is intended to permit neurogenesis and cardiogenesis without further lineage restriction, favoring aggregate-derived signaling factors and self-organization. Timing: 18+ days: The maximal duration for continued maintenance and development of EMLOC analysis has not yet been determined beyond 25 d from induction. At 7 d post-aggregation, collect aggregates in a 15 mL tube and let settle at 37° C. for 10 min. Remove medium and rinse with HBSS (CM-free). Let re-settle and remove the HBSS. Exchange the medium to non-supplemented N2B27 and re-distribute EMLOCs evenly to new cultureware freshly treated with Anti-Adherence Rinsing Solution. Place on orbital shaker at 75 rpm clockwise in a humidified incubator with 5% CO₂. Replenish N2B27 basal media every 3-5 d as needed to maintain the maturing EMLOCs for the remainder of the protocol.

### Medium, Composition, Concentration, and Configuration Table:

| Medium | Composition | Concentration | | Configuration |
|---|---|---|---|---|
| Induction | FGF2 | 40 | ng/mL | 48 hours total, with |
| Medium | CHIR 99021 | 3 | μM | a change of |
| | | | | medium after 24 h |
| EMLOC | FGF2 | 10 | ng/mL | 48 hours total, with |
| Polarization | IGF1 | 2 | ng/mL | a change of half |
| Medium | HGF | 2 | ng/mL | medium after 24 h |
| | ROCK inhibitor | 50 | μM | |
| | Y-27632 | | | |
| Cardiac | VEGF | 5 | ng/mL | 120 hours total (5 |
| Induction | FGF2 | 30 | ng/mL | days), with change |
| Medium | Ascorbic Acid | 0.5 | mM | in medium as |
| | | | | needed |

Expected Outcomes: The EMLOC formation and multi-lineage differentiation protocol occurs in four general stages (FIG. Ss1). Stage 1 of hiPSC induction to EMLOC-primed cells is performed using intact 2D colonies beginning at ˜50-60% confluency (FIGS. Ss2B and Ss2C). The induction period using N2B27 supplemented with CHIR 99021 and FGF2 occurs over 48 h, however, may require longer exposure to between 48-72 h depending on cell line. By 48 h, optimal colony induction by visual inspection is characterized by slight raised-edge character just as cells begin to migrate away from the colony border (FIG. Ss2D). Induced cells should co-express neuroectodermal biomarker SOX2 and mesendodermal biomarker FOXA2 by immunofluorescence (FIG. Ss3A). At this point, 2D cultures are primed and ready to proceed to Stage 2, transition to shaking culture and EMLOC early polarization.

Stage 2 is characterized by exposure to FGF2, IGF1 and HGF in N2B27 basal medium for 48 h during aggregation of 2D primed colonies to 3D spherical aggregates. This necessitates dissociation of primed colonies to a single cell suspension using diluted Accutase. Prior to aggregation on orbital shaking culture in low-adhesion 6-well plates, a satisfactory single cell suspension should be verified by visual inspection using a tissue culture microscope. Single cells are applied at 2-4×10⁶ cells/well depending on cell line in 2 mL of Aggregation Medium. After 24 h on the orbital shaker (80 rpm), spherical aggregates with size distribution of ˜50-100 μm should be present (FIG. Ss2E). A critical step is to ensure appropriate size, and therefore cell number per initial aggregate (50-100 cells). Aggregates at 24 h should retain co-expression of SOX2 and FOXA2 as shown (FIG. Ss3B), but do not yet express GATA6. A one-half volume media change to Polarization Medium (no ROCK inhibitor) is made at 24 h with attempts to maintain steady-state levels of the recombinant proteins FGF2, IGF1 and HGF. After an additional 24 h (48 h post-aggregation), aggregates should have increased by ˜50% in size before proceeding to Stage 3.

At Stage 3, early EMLOC aggregates undergo cardiac induction by exposure to the angiocrine and cardiogenic factors FGF2, VEGF and ascorbic acid in N2B27 basal medium. By 48 h after exposure to the Cardiac Induction Medium (day 4 post-aggregation), polarized EMLOCs are oblong with cardiac crescent structures evident on visual inspection (FIG. Ss2F) and identified by immunofluorescence of the biomarkers GATA4 and cTnT (FIG. Ss3C). The cardiac crescent elongates to become the contractile chamber-like structure by day 7 (FIGS. Ss2G and Ss3D), which retains GATA4 and cTnT expression in addition to GATA6. The border of the cardiac crescent with non-cardiac EMLOC components can also be visualized by the N-cadherin, CDH2. Contractility can be quantified in culture (beat frequency, percentage of beating aggregates) and by live-cell calcium imaging with Fluo-4 AM dye (FIG. Ss2H). Once contractile chamber-like structures emerge and are frequently identified in culture, cardiac induction factors can be withdrawn. Importantly, early neurogenesis from SOX2+ neural rosettes distal to the cardiac compartment should be occurring at day 7 and can be identified as early as day 4 (FIGS. Ss3C and Ss3F). At this stage, newly differentiated neurons are diverted away from the cardiac region, potentially due to influence by extracellular matrix-derived factors (FIG. Ss3F). Self-organized anterior foregut tissue (FOXA2, CDH1) posterior to chamber-like structures may also be identified during Stage 3. Beyond day 7, cultures are maintained in N2B27 basal medium without supplements to permit further EMLOC multi-lineage differentiation, cellular diversification, and maturation (Stage 4).

The contractile, polarized EMLOCs undergo robust neurogenesis during Stage 4 and were maintained in the original publication to day 25. By this time point, the cTnT+ cardiac region should be populated by a subset of the neurons generated (FIG. Ss3H). Throughout this stage of maturation, further events with developmental relevance can be observed such as expansion and consolidation of the multi-layered chamber wall with ongoing morphogenesis (FIG. Ss2I). The range of lineages present in EMLOCs along with cellular diversity and maturation state can be interrogated by immunofluorescence in addition to single cell RNA-Seq. We originally performed the RNA-Seq at two time points (days 7 and 16). In our dataset, the predominant lineages to be expected are cardiac (cardiomyocytes, epicardium, cardiac fibroblasts, etc.), vascular endothelial, trunk nervous tissue (spinal cord neural progenitors, peripheral neurons, Schwann cell glia), anterior foregut, and genitourinary epithelium with intermediate mesoderm-derived metanephric mesenchyme. In contrast to EMLOs that primarily generate motor neurons, EMLOCs generate predominantly autonomic neurons (˜70%; PHOX2B/ASCL1) with a subset of peripheral sensory (25%; POU4F1) and motor neurons (5%; MNX1). Extensive gene biomarker lists based on cell stage and lineage are also provided in our original manuscript related to this protocol (Olmsted and Paluh, 2022). The protocol for dissociation of EMLOCs to single cells for sequencing is also described therein.

This protocol describes the generation of EMLOCs with the Hispanic-Latino H3.1.1 hiPSC line. In Olmsted and Paluh (2021), we previously demonstrated the use of this line and others to reproducibly generate enteric gut formation with neural integration in an elongating multi-lineage organized (EMLO) gastruloid model (Olmsted and Paluh, 2021). In Tomov et al. (2016) we comprehensively compared multiple ethnically-diverse hiPSC lines generated in collaboration with our laboratory (Chang et al., 2015), including cardiac differentiation and narrow effective windows in CHIR 99021 concentration gradient that are cell-line specific (Tomov et al., 2016). These publications may be helpful when replicating the protocol with a new hiPSC line, ideally at low passage number.

This study applies a unique reagent that is a Hispanic-Latino low passage hiPSC line previously generated and initially characterized with other ethnically-diverse hiPSC lines (Chang et al., 2015; Tomov et al., 2016). The ethnically diverse hiPSC lines are being made available via WiCell (Madison, Wisconsin).

The consistent lifelong critical functioning of the adult human heart is established during embryonic development in a process known as cardiogenesis. EMLOCs provide the first detailed insights into integrated neurogenesis and cardiogenesis in a human gastruloid developmental model. The complex process of cardiogenesis requires short-range interactions with surrounding tissues and occurs in conjunction with long-range input by neurons through progressive innervation (Harvey, 2002; Hasan, 2013). As the first organ to function in the embryo, the developing heart begins to supply blood to the growing fetal brain as a closed tube, even before undergoing dramatic structural reorganization and maturation into septated chambers with outflow tracts. Such complexity and dependence on multiple non-cardiac tissue inputs has made it difficult to recapitulate human heart development using traditional in vitro models, requiring instead refined gastruloid technologies.

The intracardiac nervous system is sometimes colloquially referred to as the “brain within the heart” (Campos et al., 2018). Using sophisticated methodologies such as optogenetic stimulation, the role of peripheral cardiac neural circuitry in pacemaking and conduction is beginning to be understood (Rajendran et al., 2019; Fedele and Brand, 2020). Innervation of the heart in vivo is predominately autonomic, where sympathetic neurons can directly innervate working cardiomyocytes in the ventricular wall, and are networked as so-called ganglionated plexuses (Zaglia et al., 2017). The neurons that begin to develop in EMLOCs at the time when spontaneous contractility is first observed (˜day 7) are unlikely to substantially contribute to contractile function, since at this stage they are relatively few in number and do not project into the cardiogenic region. This is consistent with in vivo development where contractility of the heart tube occurs prior to innervation that is established later (George et al., 2020). However, organized neuronal networks resembling ganglionated plexuses were observed as EMLOCs progressively matured. At the day 7 time point, neurons were localized distantly from the cardiogenic region, before expanding significantly in number to migrate, embrace and populate the myocardial layer over time. One potential explanation is a microenvironment switch from axon-repulsive to axon-permissive as ECM in the cardiac jelly is degraded. The ECM-rich cardiac jelly in vivo contains chondroitin sulfate proteoglycans and other components known to exert repulsive or pausing effects on axons during navigation and regeneration (Tom et al., 2004). Degradation of the cardiac jelly during development is physiologic and required for normal cardiac chamber morphogenesis (Kim et al., 2018). Differential regulation of SEMA3A expression may also play a role. Within this framework, our data support the adherence of EMLOC events to physiologic spatiotemporal developmental processes for establishing contractile chambers with supplied neurons (Hasan, 2013; George et al., 2020). Neuromuscular interactions between cardiac innervating neurons and cardiomyocytes at the “neuro-cardiac junction” remains poorly understood (Zaglia et al., 2017) including biomarkers. Synapses with cardiomyocytes are postulated to be mediated through an alternate structure other than the nAChR machinery in skeletal neuromuscular junctions (Sargent and Garrett, 1995). Traditional 2D hiPSC differentiation protocols that generate human neurons and cardiomyocytes separately and then co-culture these cells to obtain structural and functional detail are typically used to study innervation, as has been done for the skeletal muscle neuromuscularjunction (Darabid et al., 2014; Steinbeck et al., 2016). EMLOCs are expected to provide a developmental and spatiotemporal perspective of the neuro-cardiac junction.

Efforts to study heart development and function using human cells also focus on separate cardiac mechanisms and include combined tissue engineering platforms and solutions (Ma et al., 2015; Macqueen et al., 2018). Such top-down human intervention of biofabricated tissues and organs has not yet achieved developmentally patterned neuronal innervation (Das et al., 2020), but may benefit from this EMLOC study. Gastruloid models that more closely mimic embryogenesis are an exciting alternative to achieve and study organogenesis (van den Brink et al., 2014; Beccari et al., 2018; Moris et al., 2020). A recent study with mESCs made significant advances and achieved early key features of cardiogenesis (Rossi et al., 2021). Our developmental model of human cardiogenesis in gastruloids further advances cardiac models by including neuronal co-development and association with the myocardium. The scRNAseq analysis that indicates that we have established multiple prerequisites for innervation. In our previous study of EMLO gastruloids that generated CNS and PNS integration with mixed lineage trunk identity (Olmsted and Paluh, 2020a), we achieved self-organized spinal neurons, neural crest, and a primitive gut tube surrounded by splanchnic mesenchyme, thereby providing much of the ideal cardiogenic microenvironment. By modifying the EMLO protocol (Olmsted and Paluh, 2020b) to include pro-cardiogenic and angiogenic factors, VEGF and ascorbic acid, that were applied in the mESC in vitro cardiogenesis study (Rossi et al., 2021), we achieved coupled cardiogenesis and neurogenesis. Through comprehensive biomarker analysis and live cell calcium imaging, it is demonstrated here that EMLOCs recapitulate numerous key features of human cardiogenesis including cardiac crescent transformation into the contractile heart tube, cardiomyocyte differentiation versus remodeling phases, and formation of chamber and outflow tract-like structures. Cardiogenesis occurs anterior to primitive gut tube-like endodermal cells that in vivo are thought to be required (Nascone and Mercola, 1995; Schultheiss et al., 1995; Varner and Taber, 2012; Anderson et al., 2016; Kidokoro et al., 2018; Han et al., 2020). By comparing calcium transients per minute in the EMLOC system versus the mESC cardiogenesis study in gastruloids, a similar species-specific ratio to that between the resting adult heart rate in human versus mouse (˜8× higher in the murine model) that have similar processes of cardiogenesis (Krishnan et al., 2014) was observed.

EMLOCs will open new opportunities to study fundamental questions on neuromodulation of contracting cardiomyocytes with relevance to neurocardiogenic syncope and other neural-based arrhythmia pathologies (Ashton et al., 2018). As well, such a neuro-cardiac model system is expected to provide fundamental insights into the pathophysiology of congenital heart disease and potential treatments in addition to viral infection studies and in vitro pharmacotherapy testing and discovery. As a drastically needed component of in vitro stem cell systems, innervation in non-neural tissue, organ, and embryo models (Das et al., 2020; Sahu and Sharan, 2020) is beginning to be achieved in EMLO and EMLOC gastruloids to advance innervation research. Embodiments, of the present disclosure including models will have broad biomedical relevance for neuro-cardiac development and human organ innervation initiatives.

In this work a gastruloid model enabling co-development and self-integration of human neuronal and cardiac tissue precursors in multicellular and multi-lineage context was provided. These results extend previous work with neurogenesis and gut development in EMLO gastruloids to promote concomitant cardiogenesis that recapitulates multiple key features of in vivo heart development. A limitation of this study is the use of one hiPSC line and ability to evaluate only two developmental time points by scRNAseq, of the numerous stages analyzed and described. Nine lines previously evaluated for EMLO formation revealed reproducibility of structural organization and cell types but with differences in efficiency between lines that can be optimized (Olmsted and Paluh, 2021a; 2021b). The EMLOC system represents the first developmentally based human neuro-cardiac model that can be applied to advance knowledge. EMLOCs neurons are co-produced endogenously within the same gastruloid and in the context of the developing heart. This is as opposed to separate generation of neural or cardiac cells followed by combination by fusion or co-cultures (sometimes referred to as assembloids). The EMLOC generates a more natural path to neuron integration with the heart, both spatially and temporally, that mimics ex vivo what is seen in human embryos.

The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described herein, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described herein. Accordingly, the scope of the present disclosure includes all modifications and equivalents of the subject matter recited in the claims appended hereto as permitted by applicable law. Moreover, any combination of the above-described elements in all possible variations thereof is encompassed by the scope of the present disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A method of generating elongating multi-lineage organized cardiac (EMLOC) gastruloids, comprising: contacting a colony of pluripotent stem cells with hepatocyte growth factor (HGF), insulin-like growth factor-1 (IGF-1), fibroblast growth factor-2 (FGF-2) under culture conditions for a first duration to form a first plurality of gastruloids; and contacting the first plurality of gastruloids with vascular endothelial growth factor (VEGF), fibroblast growth factor-2 (FGF-2), and ascorbic acid under culture conditions for a second duration to form EMLOC gastruloids.

2. The method of claim 1, wherein the pluripotent stem cells are human induced pluripotent stem cells having been derived from fibroblasts.

3. The method of claim 1, wherein the growth factor (HGF), insulin-like growth factor (IGF), and fibroblast growth factor (FGF) are provided in an effective amount to cause the colony of pluripotent stem cells to form a first plurality of gastruloids.

4. The method of claim 1, wherein the vascular endothelial growth factor (VEGF), fibroblast growth factor (FGF), and ascorbic acid are provided in an effective amount to stimulate the first plurality of gastruloids to form EMLOC gastruloids.

5. The method of claim 1, wherein pluripotent stem cells are contacted under culture conditions with 2 ng/ml hepatocyte growth factor (HGF), 2 ng/ml insulin-like growth factor (IGF), 10 ng/ml fibroblast growth factor (FGF) for a first duration, and 5 ng/ml vascular endothelial growth factor (VEGF), 30 ng/ml fibroblast growth factor (FGF), and 0.5 mM ascorbic acid for a second duration.

6. The method of claim 1, wherein the contacting occurs in an environment characterized as cardiac permissive.

7. The method of claim 1, wherein the colony of human pluripotent stem cells is characterized as trunk-biased stem cells.

8. The method of claim 1, wherein the first duration is 24 hours, and the second duration is 2-7 days.

9. The method of claim 1, wherein the colony of human pluripotent stem cells are trunk-biased stem cells.

10. An EMLOC gastruloid formed by the method of claim 1.

11. An EMLOC gastruloid, comprising a three-dimensional structure comprising contractile innervated tissue.

12. A method of generating a contractile innervated human heart tissue or organ, comprising: stimulating an exogenous population of stem cells to divide and differentiate to an innervated cardiac cells by contacting the exogenous population of stem cells with hepatocyte growth factor (HGF), insulin-like growth factor-1 (IGF-1), fibroblast growth factor-2 (FGF-2) under culturing conditions for a first duration to form a first plurality of gastruloids; and contacting the first plurality of gastruloids with vascular endothelial growth factor (VEGF), fibroblast growth factor-2 (FGF-2), and ascorbic acid under culture conditions for a second duration.

13. A method of generating an organized co-developed neuro-cardiac gastruloid, comprising: contacting an exogenous population of stem cells with an effective amount of hepatocyte growth factor (HGF), insulin-like growth factor (IGF-1), fibroblast growth factor (FGF-2) for a first duration, and subsequently contacting the exogenous population of stem cells with an effective amount of vascular endothelial growth factor (VEGF), fibroblast growth factor (FGF-2), and ascorbic acid for a second duration.

14. The method of claim 13, wherein prior to contacting, the exogenous population of stem cells are characterized as adherent, and contacted with a biologically active stem cell differentiation and reprogramming reagent and a fibroblast growth factor 2 (FGF-2).

15. A method of preparing heart-like tissue, comprising: contacting a colony of human pluripotent stem cells with hepatocyte growth factor (HGF), insulin-like growth factor-1 (IGF-1), fibroblast growth factor-2 (FGF-2) under culture conditions for a first duration; and subsequently contacting the colony of human pluripotent stem cells with vascular endothelial growth factor (VEGF), fibroblast growth factor-2 (FGF-2), and ascorbic acid under culture conditions for a second duration to form heart-like tissue.

16. The method of claim 15, wherein the heart-like tissue is characterized as comprising a plurality of EMLOC gastruloids.

17. The method of claim 15, wherein the colony of human pluripotent stem cells are contacted with an effective amount of hepatocyte growth factor (HGF), insulin-like growth factor-1 (IGF-1), fibroblast growth factor-2 (FGF-2), and combinations thereof.

18. The method of claim 15, wherein the colony of human pluripotent stem cells are contacted with an effective amount of vascular endothelial growth factor (VEGF), fibroblast growth factor-2 (FGF-2), ascorbic acid, and combinations thereof.

17. A heart-like tissue formed by the methods of claim 15.

18. The heart-like tissue of claim 17, wherein the heart-like tissue is characterized as synthetic or non-naturally occurring.

19. The heart-like tissue of claim 17, wherein the heart-like tissue is characterized as innervated and contractile.

20. A method for using the elongating multi-lineage organized cardiac (EMLOC) gastruloids of claim 1, comprising: dissociating cells of the elongating multi-lineage organized cardiac gastruloids to obtain dissociated cells, and reseeding the dissociated cells onto mammalian scaffold material to initiate structures similar in EMLOCs for cardiogenesis or innervation.

21. A method for using the elongating multi-lineage organized cardiac (EMLOC) gastruloids of claim 1, comprising: utilizing mammalian cells and initiating structures similar in EMLOCs for cardiogenesis or innervation.
